(19) United States
(12) Patent Application Publication
(10) Pub. No.: US 2025/0320479 A1
(43) Pub. Date: Oct. 16, 2025

(54) MUTANTS HAVING EFFICIENT TRANSFRUCTOSYLATION ACTIVITY

(71) Applicant: REVELATIONS BIOTECH PRIVATE LIMITED, Hyderabad (IN)

(72) Inventors: Ravi Chandra BEERAM, Hyderabad (IN); Dipanwita SINHA, Hyderabad (IN); Musuku Bharath BABU, Hyderabad (IN); Deepika SUNKARA, Hyderabad (IN); Shraddha LAHOTI, Hyderabad (IN); Anupa ANIRUDHAN, Hyderabad (IN)

(21) Appl. No.: 18/712,242

(22) PCT Filed: Nov. 22, 2022
(86) PCT No.: PCT/IN2022/051021

§ 371 Date: May 21, 2024

(30) Foreign Application Priority Data
Nov. 22, 2021 (IN) 202141053708

(51) Int. Cl. C12N 9/26; C12N 15/81; G16B 20/20

(52) U.S. Cl. CPC C12N 9/2431; C12N 15/815; C12Y 302/01026; G16B 20/20

(57) ABSTRACT

The present invention relates to improved microbial enzymes with transfructosylation activity for efficient and cost-effective production of fructo-oligosaccharides. More specifically, the invention is directed towards obtaining mutant FTase family of genes from genus Aspergillus. Nucleic acids, peptide sequences, mutant proteins, vectors and host cells for recombinant expression of novel FTases are also provided. Various mutations, such as but not limited to point mutations and deletion mutations as well as combinations thereof are presented herein. The invention also relates to a process for the expression of a novel recombinant FTase mutants as a secreted protein. The enzymes exhibit high purity after filtration, which eliminates the need for costly chromatographic procedures.

### CROSS REFERENCE TO RELATED APPLICATIONS

This application is the U.S. National Stage of PCT/IN2022/051021 with international filing date of Nov. 22, 2022, and which published as WO 2023/089639 on May 25, 2023, and which claims priority to Indian Application No. 202141053708 filed on Nov. 22, 2021, the contents of each of which are incorporated by reference in their entireties.

### SEQUENCE LISTING

A Sequence Listing accompanies this application and is submitted as an xml file of the sequence listing named “PCT2361-seql-000001.xml” which is 391,122 bytes in size and was created on Nov. 22, 2022. The sequence listing is electronically submitted via Patent Center and is incorporated herein by reference in its entirety.

### FIELD OF INVENTION

The present invention relates to the field of genetic engineering oriented to obtain improved microbial enzymes with transfructosylation activity for efficient and cost-effective production of fructo-oligosaccharides. More specifically, the invention is directed towards obtaining mutant FTase family of genes from genus Aspergillus.

### BACKGROUND

Fructose oligomers, also known as fructooligosaccharides (FOS) constitute a series of homologous oligosaccharides. Fructooligosaccharides are usually represented by the formula GFₙ and are mainly composed of 1-kestose (GF2), nystose (GF3) and β-fructofuranosylnystose (GF4), in which two, three, and four fructosyl units are bound at the β-2,1 position of glucose.

Fructooligosaccharides (FOS) are characterized by many beneficial properties such as low sweetness intensity and usefulness as a prebiotic. Due to the low sweetness intensity (about one-third to two-third as compared to sucrose) and low calorific values (approximately 0-3 kcal/g), fructooligosaccharides can be used in various kinds of food as a sugar substitute. Further, as a prebiotic, fructooligosaccharides have been reported for being used as protective agents against colon cancer, enhancing various parameters of the immune system, improving mineral adsorption, beneficial effects on serum lipid and cholesterol concentrations and exerting glycemic control for controlling obesity and diabetes (Dominguez, Ana Luisa, et al. “An overview of the recent developments on fructooligosaccharide production and applications.” Food and bioprocess technology 7.2 (2014): 324-337.) However, fructooligosaccharides are found only in trace amounts as natural components in fruits, vegetables, and honey. Due to such low concentration, it is practically impossible to extract fructooligosaccharides from food.

Attempts have been made to produce fructooligosaccharides through enzymatic synthesis from sucrose by microbial enzymes with transfructosylation activity. However, the major constraints in the previous attempts have been the lower catalytic efficiency, feedback inhibition of the enzyme by glucose leading lower FOS yields and the requirement of longer time periods for conversion of sucrose by the enzymes expressed in the recombinant host system. Further, industrial production of microbial enzymes exhibiting transfructosylation activity is challenging due to additional limitations associated with large scale expression of enzyme, enzyme stability, fermentation and purification processes.

Commercial-scale production of fructooligosaccharides requires identification and mass production of efficient enzymes. Due to the aforesaid limitations, the production of microbial enzymes with efficient transfructosylation activity is a costly affair which in-turn increases the production cost of fructooligosaccharides.

Thus, there is a long-felt and continuous need for identifying and providing efficient, cheap and industrially useful enzymes with superior transfructosylation activity, which in turn lowers the cost of production of fructooligosaccharides.

FTase family of genes/proteins includes several enzymes, such as, but not limited to β-fructofuranosidases from Aspergillus niger, fructosyltransferases from Aspergillus japonicus, Arabinanase/levansucrase/invertase from Aspergillus fijiensis and fructosyltransferase from Aspergillus aculeatus, etc. The amino acid sequences of these proteins are represented by Sequence IDs 1, 2, 3 and 4). These enzymes exhibit transfructosylation activity and are thus important in production of fructooligosaccharides.

β-fructofuranosidases and fructosyltransferases are being produced from Aspergillus genus, but there is still requirement of efficient and cost-effective enzymes. The present invention attempts to address the aforesaid problems in prior art by generating various mutant and variant strains so that better enzymes can be obtained in good quantities.

### SUMMARY OF THE INVENTION

### Technical Problem

The technical problem to be solved in this invention is to provide novel enzymes with superior transfructosylation activity.

### The Solution to the Problem

The problem has been solved by employing unique enzyme engineering as well as bio-informatics based approaches for developing mutants of FTases from genus Aspergillus.

### Overview of the Invention

The present invention relates to nucleic acids, peptide sequences, mutant proteins, vectors and host cells for recombinant expression of a novel FTases, such as, but not limited to β-fructofuranosidase or fructosyltransferase enzymes. Various mutants, such as but not limited to point mutants and deletion mutants as well as combinations thereof are presented herein. The invention also relates to a process for the expression of a novel recombinant FTase mutants as a secreted protein. The enzymes exhibits high purity after filtration, which eliminates the need for costly chromatographic procedures.

Finally, the enzymes can be used for obtaining a high yield of fructooligosaccharides.

### BRIEF DESCRIPTION OF DRAWINGS

The features of the present disclosure will become fully apparent from the following description taken in conjunction with the accompanying figures. With the understanding that the figures depict only several embodiments in accordance with the disclosure and are not to be considered limiting of its scope, the disclosure will be described further through the use of the accompanying figures.

FIG. 1 depicts the multiple sequence alignment of the native FTases from some representative Aspergillus genera.

FIG. 2a depicts the multiple sequence alignment of the native fopA gene with modified fopA gene.

FIG. 2b depicts the multiple sequence alignment of the native ft gene with modified ft gene.

FIG. 3 represents the construction scheme of pPICZαA vector.

FIG. 4 depicts the results of the restriction digestion analysis performed on the recombinant plasmid pPICZαA containing (a): V44L; (b): T155R; (c): T293W and R286V; (d): T459R; (e): R199K; (f): F182P; (g): 32-654 aa, 32-194 aa and 1-194 aa mutants.

FIG. 5 depicts the SDS-PAGE analysis for screening protein expression in Pichia pastoris recombinant strains having integration (a): pPICZαA-V44L; (b): pPICZαA-T155R; (c): pPICZαA-T293W; (d): pPICZαA-R286V; (e): pPICZαA-R199K; (f): pPICZαA-T459R; (g): pPICZαA-F182P.

FIG. 6 depicts the SDS-PAGE analysis of samples collected at different time intervals during fermentation trail of various mutant FTase proteins. (a): pPICZαA-V44L; (b): pPICZαA-T155R; (c): pPICZαA-T293W; (d): pPICZαA-R286V; (e): pPICZαA-R199K; (f): pPICZαA-T459R; (g): pPICZαA-F182P

FIG. 7 depicts TLC showing formation of FOS from sucrose by the mutant enzymes. (a): V44L; (b): T155R; (c): T293W; (d): R286V; (e): R199K; (f): T459R; (g): F182P

FIG. 8 depicts HPLC analysis chromatographs of FOS samples formed by mutant enzymes. (a): V44L; (b): T155R; (c): T293W; (d): R286V; (e): R199K; (f): T459R; (g): F182P

FIG. 9 depicts the results of the restriction digestion analysis performed on the recombinant plasmid pPICZαA containing (a): N32R, H43Y, H43V, H43L, V125F, V127A, P131Y; (b): F182R, F182A, F182D, F182V, F182T, T293F, T293H; (c): T293R, T293L, T293S, Q327L; (d): Q327N, V343F

FIG. 10 depicts the SDS-PAGE analysis for screening protein expression in Pichia pastoris recombinant strains having integration (a): pPICZαA-H43Y; (b): pPICZαA-H43L; (c): pPICZαA-Q327N; (d): pPICZαA-V343F

FIG. 11 depicts workflow to identify stabilizing point mutations in FTase

FIG. 12 depicts comparative sequences of ft gene and fopA gene

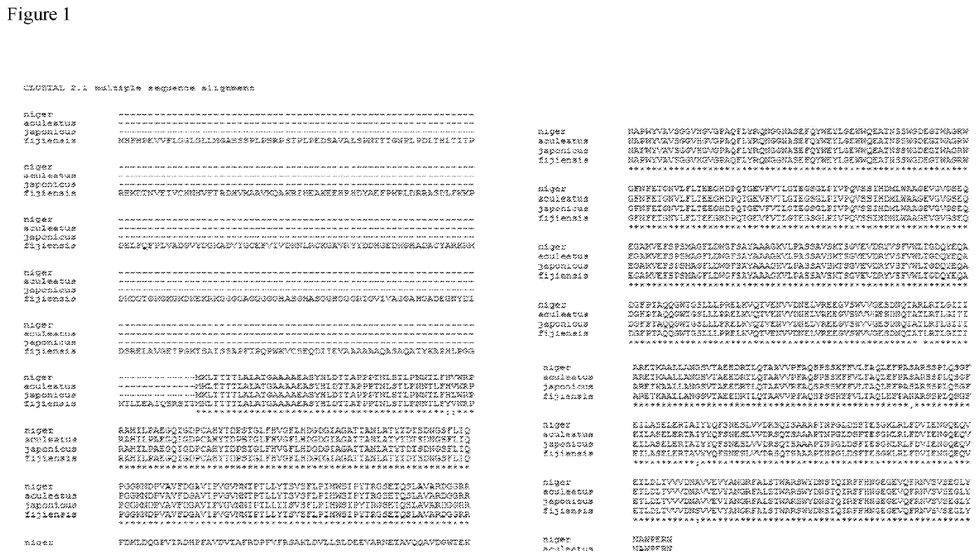

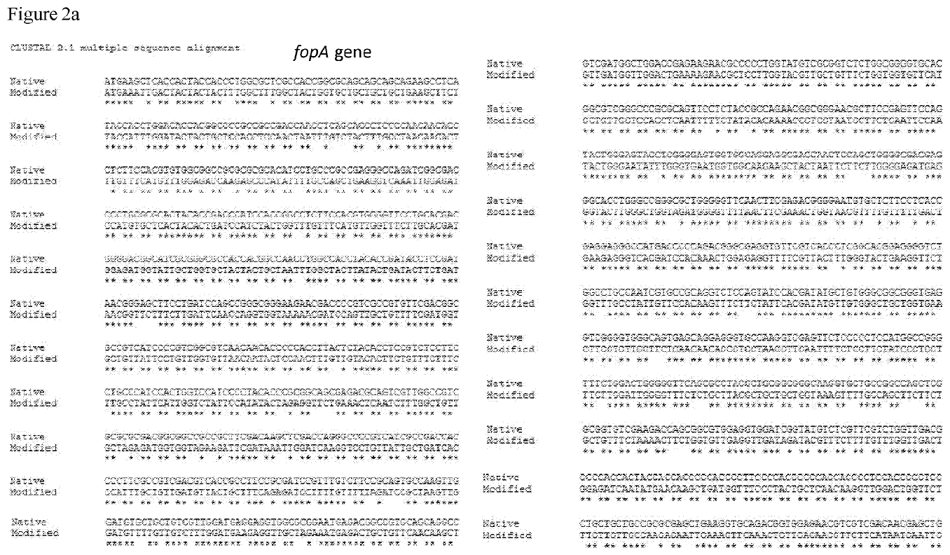

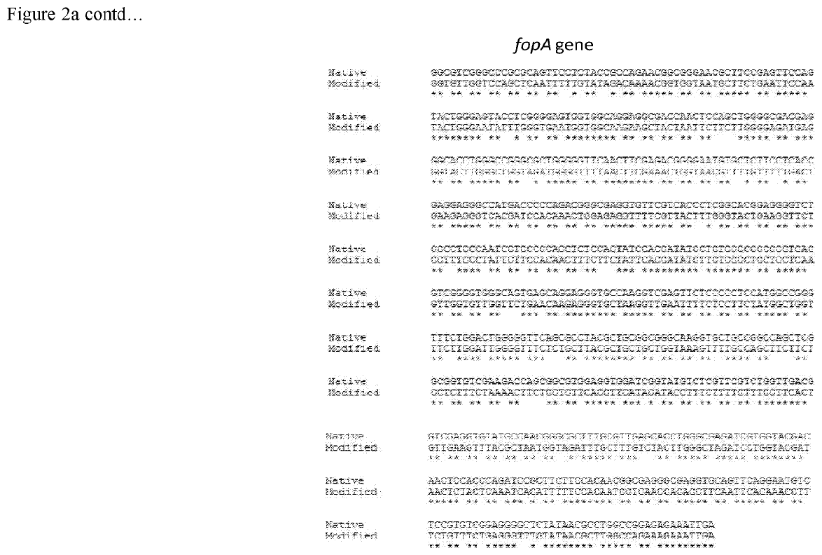

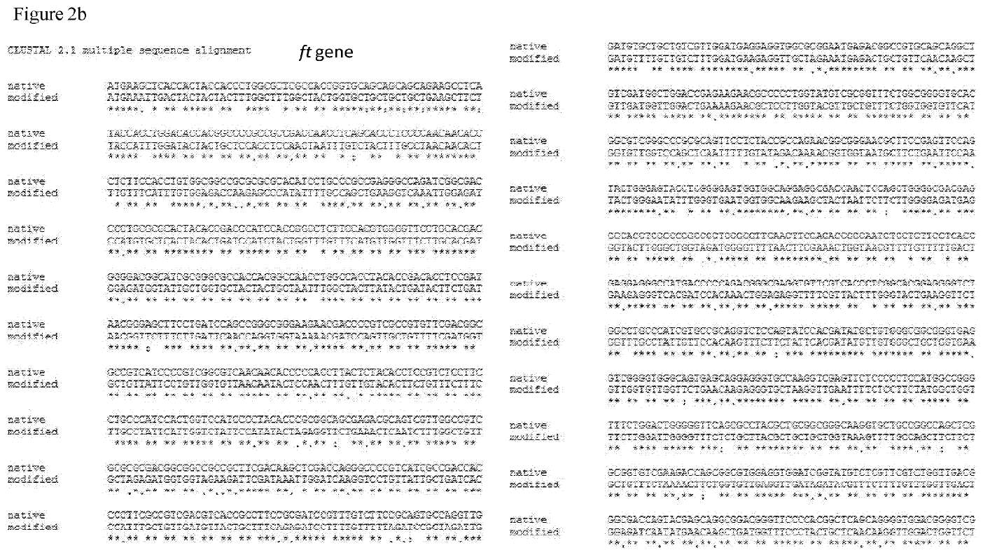

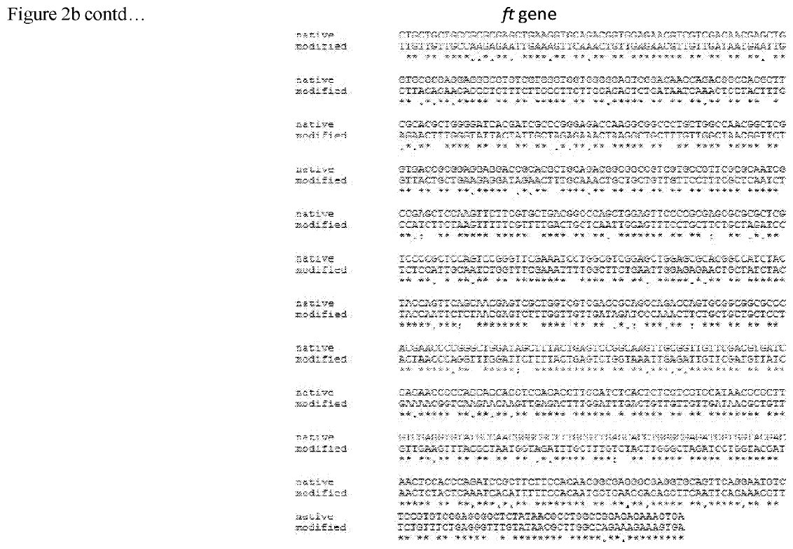

### BRIEF DESCRIPTION OF SEQUENCES AND SEQUENCE LISTING

| SEQ ID | |
|---|---|
| NO. | Brief Description |
| 1 | Sequence of the amino acid sequences of FTase from Aspergillus niger |
| 2 | Sequence of the amino acid sequences of FTase from Aspergillus japonicus |
| 3 | Sequence of the amino acid sequence of FTase from Aspergillus fijiensis |
| 4 | Sequence of the amino acid sequence of FTase from Aspergillus aculeatus |
| 5 | Nucleotide sequence of fopA gene from Aspergillus niger |
| 6 | Nucleotide sequence of codon optimized fopA gene from Aspergillus niger |
| 7 | Nucleotide sequence ft gene for fructosyltransferase from Aspergillus japonicus |
| 8 | Nucleotide sequence of codon optimized fructosyltransferase (ft) gene |
| | Aspergillus japonicus |
| 9 | Nucleotide sequence of Ftase from Aspergillus niger modified at N32R |
| 10 | Amino acid sequence of FTase from Aspergillus niger modified at N32R |
| 11 | Nucleotide sequence of Ftase from Aspergillus niger modified at H43Y |
| 12 | Amino acid sequence of FTase from Aspergillus niger modified at H43Y |
| 13 | Nucleotide sequence of Ftase from Aspergillus niger modified at H43V |
| 14 | Amino acid sequence of FTase from Aspergillus niger modified at H43V |
| 15 | Nucleotide sequence of Ftase from Aspergillus niger modified at H43L |
| 16 | Amino acid sequence of FTase from Aspergillus niger modified at H43L |
| 17 | Nucleotide sequence of Ftase from Aspergillus niger modified at V125F |
| 18 | Amino acid sequence of FTase from Aspergillus niger modified at V125F |
| 19 | Nucleotide sequence of Ftase from Aspergillus niger modified at V127A |
| 20 | Amino acid sequence of FTase from Aspergillus niger modified at V127A |
| 21 | Nucleotide sequence of Ftase from Aspergillus niger modified at P131Y |
| 22 | Amino acid sequence of FTase from Aspergillus niger modified at P131Y |
| 23 | Nucleotide sequence of Ftase from Aspergillus niger modified at T155R |
| 24 | Amino acid sequence of FTase from Aspergillus niger modified at T155R |
| 25 | Nucleotide sequence of Ftase from Aspergillus niger modified at F182P |
| 26 | Amino acid sequence of FTase from Aspergillus niger modified at F182P |
| 27 | Nucleotide sequence of Ftase from Aspergillus niger modified at F182R |
| 28 | Amino acid sequence of FTase from Aspergillus niger modified at F182R |
| 29 | Nucleotide sequence of Ftase from Aspergillus niger modified at F182A |
| 30 | Amino acid sequence of FTase from Aspergillus niger modified at F182A |
| 31 | Nucleotide sequence of Ftase from Aspergillus niger modified at F182D |
| 32 | Amino acid sequence of FTase from Aspergillus niger modified at F182D |
| 33 | Nucleotide sequence of Ftase from Aspergillus niger modified at F182V |
| 34 | Amino acid sequence of FTase from Aspergillus niger modified at F182V |
| 35 | Nucleotide sequence of Ftase from Aspergillus niger modified at F182T |
| 36 | Amino acid sequence of FTase from Aspergillus niger modified at F182T |
| 37 | Nucleotide sequence of Ftase from Aspergillus niger modified at R286V |
| 38 | Amino acid sequence of FTase from Aspergillus niger modified at R286V |
| 39 | Nucleotide sequence of Ftase from Aspergillus niger modified at T293F |
| 40 | Amino acid sequence of FTase from Aspergillus niger modified at T293F |
| 41 | Nucleotide sequence of Ftase from Aspergillus niger modified at T293H |
| 42 | Amino acid sequence of FTase from Aspergillus niger modified at T293H |
| 43 | Nucleotide sequence of Ftase from Aspergillus niger modified at T293L |
| 44 | Amino acid sequence of FTase from Aspergillus niger modified at T293L |
| 45 | Nucleotide sequence of Ftase from Aspergillus niger modified at T293R |
| 46 | Amino acid sequence of FTase from Aspergillus niger modified at T293R |
| 47 | Nucleotide sequence of Ftase from Aspergillus niger modified at T293S |
| 48 | Amino acid sequence of FTase from Aspergillus niger modified at T293S |
| 49 | Nucleotide sequence of Ftase from Aspergillus niger modified at T293W |
| 50 | Amino acid sequence of FTase from Aspergillus niger modified at T293W |
| 51 | Nucleotide sequence of Ftase from Aspergillus niger modified at Q327L |
| 52 | Amino acid sequence of FTase from Aspergillus niger modified at Q327L |
| 53 | Nucleotide sequence of Ftase from Aspergillus niger modified at Q327N |
| 54 | Amino acid sequence of FTase from Aspergillus niger modified at Q327N |
| 55 | Nucleotide sequence of Ftase from Aspergillus niger modified at V343F |
| 56 | Amino acid sequence of FTase from Aspergillus niger modified at V343F |
| 57 | Nucleotide sequence of deletion mutation corresponding to >32-654 amino acid |
| 58 | Amino acid sequence of deletion mutation corresponding to >32-654 amino acid |
| 59 | Nucleotide sequence of deletion mutation corresponding to >32-194 amino acid |
| 60 | Amino acid sequence of deletion mutation corresponding to >32-194 amino acid |
| 61 | Nucleotide sequence of deletion mutation corresponding to >1-194 amino acid |
| 62 | Amino acid sequence of deletion mutation corresponding to >1-194 amino acid |
| 63 | Nucleotide sequence of Ftase from Aspergillus niger modified at H43Y and |
| | V343F |
| 64 | Amino acid sequence of FTase from Aspergillus niger modified at H43Y and |
| | V343F |
| 65 | Nucleotide sequence corresponding to >32-654 amino acid including H43Y |
| | V343F point mutation |
| 66 | Amino acid sequence corresponding to >32-654 amino acid including H43Y |
| | V343F point mutation |
| 67 | Amino acid sequence of FTase from Aspergillus niger modified at H43R |
| 68 | Amino acid sequence of FTase from Aspergillus niger modified at H43T |
| 69 | Amino acid sequence of FTase from Aspergillus niger modified at H43M |
| 70 | Amino acid sequence of FTase from Aspergillus niger modified at H43I |
| 71 | Amino acid sequence of FTase from Aspergillus niger modified at P131W |
| 72 | Amino acid sequence of FTase from Aspergillus niger modified at T155K |
| 73 | Amino acid sequence of FTase from Aspergillus niger modified at V44L |
| 74 | Amino acid sequence of FTase from Aspergillus niger modified at R166Y |
| 75 | Amino acid sequence of FTase from Aspergillus niger modified at R166N |
| 76 | Amino acid sequence of FTase from Aspergillus niger modified at F182L |
| 77 | Amino acid sequence of FTase from Aspergillus niger modified at F182W |
| 78 | Amino acid sequence of FTase from Aspergillus niger modified at F182N |
| 79 | Amino acid sequence of FTase from Aspergillus niger modified at F182K |
| 80 | Amino acid sequence of FTase from Aspergillus niger modified at F182Q |
| 81 | Amino acid sequence of FTase from Aspergillus niger modified at R196K |
| 82 | Amino acid sequence of FTase from Aspergillus niger modified at K199R |
| 83 | Amino acid sequence of FTase from Aspergillus niger modified at H240F |
| 84 | Amino acid sequence of FTase from Aspergillus niger modified at F259W |
| 85 | Amino acid sequence of FTase from Aspergillus niger modified at V343L |
| 86 | Amino acid sequence of FTase from Aspergillus niger modified at A381R |
| 87 | Amino acid sequence of FTase from Aspergillus niger modified at A381L |
| 88 | Amino acid sequence of FTase from Aspergillus niger modified at L440R |
| 89 | Amino acid sequence of FTase from Aspergillus niger modified at Q586R |
| 90 | Amino acid sequence of FTase from Aspergillus niger modified at N32Q |
| 91 | Amino acid sequence of FTase from Aspergillus niger modified at N32V |
| 92 | Amino acid sequence of FTase from Aspergillus niger modified at H43W |
| 93 | Amino acid sequence of FTase from Aspergillus niger modified at H43E |
| 94 | Amino acid sequence of FTase from Aspergillus niger modified at H43D |
| 95 | Amino acid sequence of FTase from Aspergillus niger modified at R459T |
| 96 | Amino acid sequence of FTase from Aspergillus niger modified at H43Q |
| 97 | Amino acid sequence of FTase from Aspergillus niger modified at H43F |
| 98 | Amino acid sequence of FTase from Aspergillus niger modified at H43S |
| 99 | Amino acid sequence of FTase from Aspergillus niger modified at P131R |
| 100 | Amino acid sequence of FTase from Aspergillus niger modified at T155L |
| 101 | Amino acid sequence of FTase from Aspergillus niger modified at T155V |
| 102 | Amino acid sequence of FTase from Aspergillus niger modified at R166K |
| 103 | Amino acid sequence of FTase from Aspergillus niger modified at R166H |
| 104 | Amino acid sequence of FTase from Aspergillus niger modified at F182E |
| 105 | Amino acid sequence of FTase from Aspergillus niger modified at A381I |
| 106 | Amino acid sequence of FTase from Aspergillus niger modified at Q586F |
| 107 | Amino acid sequence of FTase from Aspergillus niger modified at N32D |
| 108 | Amino acid sequence of FTase from Aspergillus niger modified at N32I |
| 109 | Amino acid sequence of FTase from Aspergillus niger modified at N32Y |
| 110 | Amino acid sequence of FTase from Aspergillus niger modified at N654K |
| 111 | Amino acid sequence of FTase from Aspergillus niger modified at N32K |
| 112 | Amino acid sequence of FTase from Aspergillus niger modified at H43A |
| 113 | Amino acid sequence of FTase from Aspergillus niger modified at H43N |
| 114 | Amino acid sequence of FTase from Aspergillus niger modified at H43K |
| 115 | Amino acid sequence of FTase from Aspergillus niger modified at V127I |
| 116 | Amino acid sequence of FTase from Aspergillus niger modified at V127M |
| 117 | Amino acid sequence of FTase from Aspergillus niger modified at P131H |
| 118 | Amino acid sequence of FTase from Aspergillus niger modified at P131F |
| 119 | Amino acid sequence of FTase from Aspergillus niger modified at P131Q |
| 120 | Amino acid sequence of FTase from Aspergillus niger modified at T132L |
| 121 | Amino acid sequence of FTase from Aspergillus niger modified at T132M |
| 122 | Amino acid sequence of FTase from Aspergillus niger modified at T132V |
| 123 | Amino acid sequence of FTase from Aspergillus niger modified at T132I |
| 124 | Amino acid sequence of FTase from Aspergillus niger modified at T155I |
| 125 | Amino acid sequence of FTase from Aspergillus niger modified at L44V |
| 126 | Amino acid sequence of FTase from Aspergillus niger modified at T155W |
| 127 | Amino acid sequence of FTase from Aspergillus niger modified at T155Y |
| 128 | Amino acid sequence of FTase from Aspergillus niger modified at T155M |
| 129 | Amino acid sequence of FTase from Aspergillus niger modified at R166L |
| 130 | Amino acid sequence of FTase from Aspergillus niger modified at R166Q |
| 131 | Amino acid sequence of FTase from Aspergillus niger modified at H180Y |
| 132 | Amino acid sequence of FTase from Aspergillus niger modified at H180R |
| 133 | Amino acid sequence of FTase from Aspergillus niger modified at F182M |
| 134 | Amino acid sequence of FTase from Aspergillus niger modified at F182H |
| 135 | Amino acid sequence of FTase from Aspergillus niger modified at F182S |
| 136 | Amino acid sequence of FTase from Aspergillus niger modified at Y232W |
| 137 | Amino acid sequence of FTase from Aspergillus niger modified at H240R |
| 138 | Amino acid sequence of FTase from Aspergillus niger modified at H240K |
| 139 | Amino acid sequence of FTase from Aspergillus niger modified at F259Y |
| 140 | Amino acid sequence of FTase from Aspergillus niger modified at R199K |
| 141 | Amino acid sequence of FTase from Aspergillus niger modified at R286I |
| 142 | Amino acid sequence of FTase from Aspergillus niger modified at L322Y |
| 143 | Amino acid sequence of FTase from Aspergillus niger modified at I324Q |
| 144 | Amino acid sequence of FTase from Aspergillus niger modified at V343Q |
| 145 | Amino acid sequence of FTase from Aspergillus niger modified at V343K |
| 146 | Amino acid sequence of FTase from Aspergillus niger modified at V343Y |
| 147 | Amino acid sequence of FTase from Aspergillus niger modified at V343I |
| 148 | Amino acid sequence of FTase from Aspergillus niger modified at A381V |
| 149 | Amino acid sequence of FTase from Aspergillus niger modified at A381K |
| 150 | Amino acid sequence of FTase from Aspergillus niger modified at A381P |
| 151 | Amino acid sequence of FTase from Aspergillus niger modified at A381Q |
| 152 | Amino acid sequence of FTase from Aspergillus niger modified at A381T |
| 153 | Amino acid sequence of FTase from Aspergillus niger modified at A381Y |
| 154 | Amino acid sequence of FTase from Aspergillus niger modified at V382I |
| 155 | Amino acid sequence of FTase from Aspergillus niger modified at T459R |
| 156 | Amino acid sequence of FTase from Aspergillus niger modified at V382L |
| 157 | Amino acid sequence of FTase from Aspergillus niger modified at F568Y |
| 158 | Amino acid sequence of FTase from Aspergillus niger modified at Q586K |
| 159 | Amino acid sequence of FTase from Aspergillus niger modified at Q586Y |
| 160 | Amino acid sequence of FTase from Aspergillus niger modified at Q586V |
| 161 | Amino acid sequence of FTase from Aspergillus niger modified at Q586W |
| 162 | Amino acid sequence of FTase from Aspergillus niger modified at A188R |
| 163 | Amino acid sequence of FTase from Aspergillus niger modified at D191K |
| 164 | Amino acid sequence of FTase from Aspergillus niger modified at D191R |
| 165 | Amino acid sequence of FTase from Aspergillus niger modified at Q327G |
| 166 | Amino acid sequence of FTase from Aspergillus niger modified at Q327H |
| 167 | Amino acid sequence of FTase from Aspergillus niger modified at Q327M |
| 168 | Amino acid sequence of FTase from Aspergillus niger modified at K654N |
| 169 | Amino acid sequence of FTase from Aspergillus niger modified at Q406R |
| 170 | Amino acid sequence of FTase from Aspergillus niger modified at T293I |
| 171 | Amino acid sequence of FTase from Aspergillus niger modified at T293K |
| 172 | Amino acid sequence of FTase from Aspergillus niger modified at A188F |
| 173 | Amino acid sequence of FTase from Aspergillus niger modified at A188Q |
| 174 | Amino acid sequence of FTase from Aspergillus niger modified at D191E |
| 175 | Amino acid sequence of FTase from Aspergillus niger modified at D191F |
| 176 | Amino acid sequence of FTase from Aspergillus niger modified at D191Q |
| 177 | Amino acid sequence of FTase from Aspergillus niger modified at A371E |
| 178 | Amino acid sequence of FTase from Aspergillus niger modified at A371M |
| 179 | Amino acid sequence of FTase from Aspergillus niger modified at E405W |
| 180 | Amino acid sequence of FTase from Aspergillus niger modified at N290L |
| 181 | Amino acid sequence of FTase from Aspergillus niger modified at N290M |
| 182 | Amino acid sequence of FTase from Aspergillus niger modified at N290R |
| 183 | Amino acid sequence of FTase from Aspergillus niger modified at Q327A |
| 184 | Amino acid sequence of FTase from Aspergillus niger modified at Q327D |
| 185 | Amino acid sequence of FTase from Aspergillus niger modified at Q327E |
| 186 | Amino acid sequence of FTase from Aspergillus niger modified at Q327F |
| 187 | Amino acid sequence of FTase from Aspergillus niger modified at Q327R |
| 188 | Amino acid sequence of FTase from Aspergillus niger modified at Q406L |
| 189 | Amino acid sequence of FTase from Aspergillus niger modified at T293M |
| 190 | Amino acid sequence of FTase from Aspergillus niger modified at T293N |
| 191 | Amino acid sequence of FTase from Aspergillus niger modified at T293Q |
| 192 | Amino acid sequence of FTase from Aspergillus niger modified at T293V |
| 193 | Amino acid sequence of FTase from Aspergillus niger modified at T293Y |
| 194 | Amino acid sequence of FTase from Aspergillus niger modified at A188H |
| 195 | Amino acid sequence of FTase from Aspergillus niger modified at A188K |
| 196 | Amino acid sequence of FTase from Aspergillus niger modified at D191A |
| 197 | Amino acid sequence of FTase from Aspergillus niger modified at D191N |
| 198 | Amino acid sequence of FTase from Aspergillus niger modified at D191W |
| 199 | Amino acid sequence of FTase from Aspergillus niger modified at A371L |
| 200 | Amino acid sequence of FTase from Aspergillus niger modified at A371V |
| 201 | Amino acid sequence of FTase from Aspergillus niger modified at E405F |
| 202 | Amino acid sequence of FTase from Aspergillus niger modified at E405G |
| 203 | Amino acid sequence of FTase from Aspergillus niger modified at E405L |
| 204 | Amino acid sequence of FTase from Aspergillus niger modified at E405N |
| 205 | Amino acid sequence of FTase from Aspergillus niger modified at F118Y |
| 206 | Amino acid sequence of FTase from Aspergillus niger modified at L78R |
| 207 | Amino acid sequence of FTase from Aspergillus niger modified at L78W |
| 208 | Amino acid sequence of FTase from Aspergillus niger modified at N290K |
| 209 | Amino acid sequence of FTase from Aspergillus niger modified at N290Q |
| 210 | Amino acid sequence of FTase from Aspergillus niger modified at Q327C |
| 211 | Amino acid sequence of FTase from Aspergillus niger modified at Q327I |
| 212 | Amino acid sequence of FTase from Aspergillus niger modified at Q327K |
| 213 | Amino acid sequence of FTase from Aspergillus niger modified at Q327V |
| 214 | Amino acid sequence of FTase from Aspergillus niger modified at Q406K |
| 215 | Amino acid sequence of FTase from Aspergillus niger modified at Q406M |
| 216 | Amino acid sequence of FTase from Aspergillus niger modified at S329N |
| 217 | Amino acid sequence of FTase from Aspergillus niger modified at S329T |
| 218 | Amino acid sequence of FTase from Aspergillus niger modified at Y404W |
| 219 | Amino acid sequence for FAK alpha factor signal peptide |
| 220 | Amino acid sequence for AT alpha factor signal peptide |
| 221 | Amino acid sequence for aa alpha amylase signal peptide |
| 222 | Amino acid sequence for GA- glucoamylase signal peptide |
| 223 | Amino acid sequence for IN- Innulinase signal peptide |
| 224 | Amino acid sequence for IV- Invertase signal peptide |
| 225 | Amino acid sequence for KP killer protein signal peptide |
| 226 | Amino acid sequence for LZ- Lysozyme signal peptide |
| 227 | Amino acid sequence for SA- serum Albumin signal peptide |
| 228 | Amino acid sequence for FAK alpha factor- LEKR signal peptide |
| 229 | Amino acid sequence for FAKS alpha factor full- LEKR signal peptide |
| 230 | Amino acid sequence for AT alpha factor_T LEKR signal peptide |
| 231 | Amino acid sequence for AA- Alpha amyalse- LEKR signal peptide |
| 232 | Amino acid sequence for GA Glucoamylase- LEKR signal peptide |
| 233 | Amino acid sequence for 233- Innulinase- LEKR signal peptide |
| 234 | Amino acid sequence for IV- Invertase- LEKR signal peptide |
| 235 | Amino acid sequence for KP killer protein- LEKR signal peptide |
| 236 | Amino acid sequence for LZ-Lysozyme- LEKR signal peptide |
| 237 | Amino acid sequence for SA- Serum albumin- LEKR signal peptide |
| 238 | Amino acid sequence for bioactive fragment containing 57-62 amino acid |
| 239 | Amino acid sequence for bioactive fragment containing 119-132 amino acid |
| 240 | Amino acid sequence for bioactive fragment containing 320-330 amino acid |
| 241 | Amino acid sequence for bioactive fragment containing 401-416 amino acid |

### Definitions

Unless defined otherwise, all technical and scientific terms used herein have the same meaning as commonly understood by one of ordinary skill in the art to which the methods belong. Although any vectors, host cells, methods and compositions similar or equivalent to those described herein can also be used in the practice or testing of the vectors, host cells, methods and compositions, representative illustrations are now described.

Where a range of values are provided, it is understood that each intervening value between the upper and lower limit of that range and any other stated or intervening value in that stated range, is encompassed within by the methods and compositions. The upper and lower limits of these smaller ranges may independently be included in the smaller ranges and are also encompassed within by the methods and compositions, subject to any specifically excluded limit in the stated range. Where the stated range includes one or both of the limits, ranges excluding either or both of those included limits are also included in the methods and compositions.

It is appreciated that certain features of the methods, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other embodiments without departing from the scope or spirit of the present methods. Any recited method can be carried out in the order of events recited or in any other order that is logically possible.

The term “host cell(s)” includes an individual cell or cell culture which can be, or has been, a recipient for the subject of expression constructs. Host cells include progeny of a single host cell. Host cells for the purposes of this invention refers to any strain of Pichia pastoris which can be suitably used for the purposes of the invention. Examples of strains that can be used for the purposes of this invention include wild type, mut+, mut S, mut− strains of Pichia such as KM71H, KM71, SMD1168H, SMD1168, GS115, X33.

The term “recombinant strain” or “recombinant host cell(s)” refers to a host cell(s) which has been transfected or transformed with the expression constructs or vectors of this invention.

The term “expression vector” refers to any vector, plasmid or vehicle designed to enable the expression of an inserted nucleic acid sequence following transformation into the host.

The term “promoter” refers to DNA sequences that define where transcription of a gene begins. Promoter sequences are typically located directly upstream or at the 5′ end of the transcription initiation site. RNA polymerase and the necessary transcription factors bind to the promoter sequence and initiate transcription. Promoters can either be constitutive or inducible promoters. Constitutive promoters are the promoter which allows continual transcription of its associated genes as their expression is normally not conditioned by environmental and developmental factors. Constitutive promoters are very useful tools in genetic engineering because constitutive promoters drive gene expression under inducer-free conditions and often show better characteristics than commonly used inducible promoters. Inducible promoters are the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cell type.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter).

The term “transcription” refers to the process of making an RNA copy of a gene sequence. This copy, called a messenger RNA (mRNA) molecule, leaves the cell nucleus and enters the cytoplasm, where it directs the synthesis of the protein, which it encodes.

The term “translation” refers to the process of translating the sequence of a messenger RNA (mRNA) molecule to a sequence of amino acids during protein synthesis. The genetic code describes the relationship between the sequence of base pairs in a gene and the corresponding amino acid sequence that it encodes. In the cell cytoplasm, the ribosome reads the sequence of the mRNA in groups of three bases to assemble the protein.

The term “expression” refers to the biological production of a product encoded by a coding sequence. In most cases, a DNA sequence, including the coding sequence, is transcribed to form a messenger-RNA (mRNA). The messenger-RNA is then translated to form a polypeptide product that has a relevant biological activity. Also, the process of expression may involve further processing steps to the RNA product of transcription, such as splicing to remove introns, and/or post-translational processing of a polypeptide product.

The term “modified polypeptide/polynucleotide” as used herein is used to refer to a polypeptide or polynucleotide encoding FTase selected from but not limited to 0-fructofuranosidase and/or fructosyltransferase mutants fused to a signal peptide. The functional variant includes any nucleic acid having substantial or significant sequence identity or similarity to the β-fructofuranosidase and/or fructosyltransferase mutants, as described herein which retains the biological activities of the same.

The term “variant” as used herein in reference to pre-cursor peptides/proteins refers to peptides with amino acid substitutions, additions, deletions or alterations that do not substantially decrease the activity of the signal peptide or the enzyme. Variants include a structural as well as functional variants. The term variant also includes the use of a substituted amino acid in place of an unsubstituted parent amino acid.

Amino acid substitution tables providing functionally similar amino acids are well known to one of ordinary skill in the art. The following six groups (Table 1) are examples of amino acids that are considered to be variants for one another:

TABLE 1
Amino acid substitution table

| | Amino acids |
|---|---|
| Group 1 | Alanine (A), Serine (S), Threonine (T), |
| | Glycine (G), Proline (P) |
| Group 2 | Aspartic acid (D), Glutamic acid (E), |
| | Asparagine (N), Glutamine (Q) |
| Group 3 | Arginine (R), Lysine (K), Histidine (H) |
| Group 4 | Isoleucine (I), Leucine (L), |
| | Methionine (M), Valine (V) |
| Group 5 | Phenylalanine (F), Tyrosine (Y), Tryptophan (W) |
| Group 6 | Cysteine (C) |

The term “point mutation” refer to a large category of mutations that describe a change in single nucleotide of DNA, such that the nucleotide is switched for another nucleotide, or that nucleotide is deleted, or a single nucleotide is inserted into the DNA that causes that DNA to be different from the normal or wild type gene sequence.

The term “deletion mutation” refers to a type of mutation involving the loss of genetic material. It can be small, involving a single missing DNA base pair, or large, involving a piece of a chromosome.

The terms “polypeptide”, “peptide” and “protein” are used interchangeably herein to refer to two or more amino acid residues joined to each other by peptide bonds or modified peptide bonds. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. “Polypeptide” refers to both short chains, commonly referred to as peptides, oligopeptides or oligomers, and to longer chains, generally referred to as proteins. Polypeptides may contain amino acids other than the 20 gene-encoded amino acids. Likewise, “protein” refers to at least two covalently attached amino acids, which includes proteins, polypeptides, oligopeptides, and peptides. A protein may be made up of naturally occurring amino acids and peptide bonds, or synthetic peptidomimetic structures. Thus “amino acid”, or “peptide residue”, as used herein means both naturally occurring and synthetic amino acids. “Amino acid” includes imino acid residues such as proline and hydroxyproline. The side chains may be in either the (R) or the (S) configuration.

The term “signal peptide” or “signal peptide sequence” is defined herein as a peptide sequence usually present at the N-terminal end of newly synthesized secretory or membrane polypeptides which directs the polypeptide across or into a cell membrane of the cell (the plasma membrane in prokaryotes and the endoplasmic reticulum membrane in eukaryotes). It is usually subsequently removed. In particular said signal peptide may be capable of directing the polypeptide into a cell's secretory pathway.

The term “precursor peptide” as used herein refers to a peptide comprising a signal peptide (also known as leader sequences) operably linked to the respective FTases from genus Aspergillus. The signal peptides are cleaved off during post-translational modifications inside the Pichia host cells and the mature FTase mutants are released into the medium.

### DETAILED DESCRIPTION OF THE INVENTION

The present invention is described by the following specific embodiments. Those with ordinary skill in the art can readily understand the other advantages and functions of the present invention after reading the disclosure of this specification. Various details described in this specification can be modified based on different requirements and applications without departing from the scope of the currently disclosed invention.

Unless contraindicated or noted otherwise, throughout this specification, the terms “a” and “an” mean one or more, and the term “or” means and/or.

The use of “comprise”, “comprises”, “comprising”, “contain”, “contains”, “containing”, “include”, “includes”, and “including” are not intended to be limiting. It is to be understood that both the foregoing general description and this detailed description are exemplary and explanatory only and are not restrictive.

As used herein, biotechnological terms have their conventional meaning as illustrated by the following illustrative definitions.

Unless otherwise defined, scientific and technical terms used herein shall have the meanings that are commonly understood by those of ordinary skill in the art. Further, unless otherwise required by context, singular terms shall include pluralities and plural terms shall include the singular.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

Fructosyl transferase (FT) enzyme is a member of glucose hydrolase 32 family (GH32) which catalyses the production of fructans which are fructose oligosaccharides through a retaining mechanism. Three-dimensional structure of the enzyme FT from Aspergillus japonicus (AjFT) has been solved that provided a structural basis of the substrate binding and catalysis carried out by this enzyme. Among the structures that were solved by Chuankhayan et. al. [1], the apo structure (PDB ID: 3LF7) and the kestose bound (PDB ID: 3LDR) structures of AjFT are relevant for the present study.

The present invention discloses nucleic acids, vectors and recombinant host cells for efficient production of biologically active and soluble recombinant FTases, including, but not limited to β-fructofuranosidases from Aspergillus niger, fructosyltransferases from Aspergillus japonicus, Arabinanase/levansucrase/invertase from Aspergillus fijiensis, fructosyltransferase from Aspergillus aculeatus, mutants thereof obtained from genus Aspergillus as a secreted protein. Further, the invention provides a process for commercial-scale production of recombinant β-fructofuranosidase and/or fructosyltransferase mutants. As representative, but not restrictive examples, we mention fructosyltransferase, encoded by ft gene of Aspergillus japonicus and β-fructofuranosidase, encoded by fopA gene of Aspergillus niger.

The invention contemplates a multidimensional approach for achieving a high yield of novel recombinant FTases mutants in a heterologous host. The codon optimized gene for FTase selected from but not limited to fructosyltransferase or β-fructofuranosidase, Arabinanase/levansucrase/invertase, has been modified by way of mutation for expression in Pichia pastoris. The mutations could be point mutations or deletion mutations or combinations thereof.

In an embodiment, the codon-optimized gene for β-fructofuranosidase and/or fructosyltransferase has been modified for expression in a heterologous host cell. Further, the modified gene has been fused to one or more signal peptides.

In one embodiment, the codon optimized nucleic acid encoding β-fructofuranosidase of Aspergillus niger is represented by SEQ ID NO: 6.

In one embodiment, the codon optimized nucleic acid encoding modified fructosyltransferase of Aspergillus japonicus is represented by SEQ ID NO: 8.

In another embodiment, the modified nucleic acid is fused to one or more signal peptide.

In another embodiment, the signal peptide is selected from Alpha-factor of S. cerevisiae (FAK), Alpha-factor full of S. cerevisiae (FAKS) of S. cerevisiae, Alpha factor_T of S. cerevisiae (AT), Alpha-amylase of Aspergillus niger (AA), Glucoamylase of Aspergillus awamori (GA), Inulinase of Kluyveromycesmaxianus (IN), Invertase of S. cerevisiae (IV), Killer protein of S. cerevisiae (KP), Lysozyme of Gallus gallus (LZ), Serum albumin of Homo sapiens (SA)

In another embodiment, the signal peptides are provided in the Table 2.

TABLE 2
Signal peptides

| Seq | Signal Peptides | | Length |
|---|---|---|---|
| No. | (Source) | Amino Acid Sequence | (a.a.) |
| 219 | FAK - Alpha-factor | MRFPSIFTAVLFAASSALAAPVNTTTEDE | 81 |
| | (S. cerevisiae) | TAQIPAEAVIGYSDLEGDFDVAVLPFSNS | |
| | | TNNGLLFINTTIASIAAKEEGVS | |
| 220 | AT - Alpha-factor_T | MRFPSIFTAVLFAASSALA | 19 |
| | (S. cerevisiae) | | |
| 221 | AA - Alpha-amylase | MVAWWSLFLYGLQVAAPALA | 20 |
| | (Aspergillus niger) | | |
| 222 | GA - Glucoamylase | MSFRSLLALSGLVCSGLA | 18 |
| | (Aspergillus awamori) | | |
| 223 | IN - Inulinase | MKLAYSLLLPLAGVSA | 16 |
| | (Kluyveromyces | | |
| | maxianus) | | |
| 224 | IV - Invertase | MLLQAFLFLLAGFAAKISA | 19 |
| | (S. cerevisiae) | | |
| 225 | KP - Killer protein | MTKPTQVLVRSVSILFFITLLHLVVA | 26 |
| | (S. cerevisiae) | | |
| 226 | LZ - Lysozyme | MLGKNDPMCLVLVLLGLTALLGICQG | 26 |
| | (Gallus gallus) | | |
| 227 | SA - Serum albumin | MKWVTFISLLFLFSSAYS | 18 |
| | (Homo sapiens) | | |

In another embodiment, the harvested β-fructofuranosidase of Aspergillus niger as well as fructosyltransferases from Aspergillus japonicus was characterized to identify the bioactive fragments. It was found that the following bioactive fragments of conserved FTases that account for the catalytic activities are provided in the Table 3 below:

TABLE 3
Bioactive fragments of B-fructo-
furanosidase are conserved and
accounts for the catalytic activity.

| Position | Fragment | SEQ ID Number |
|---|---|---|
| 57-62 | QIGDPC | SEQ ID NO: 238 |
| 119-132 | DGAVIPVGVNNTPT | SEQ ID NO: 239 |
| 320-330 | SGLPIVPQVS | SEQ ID NO: 240 |
| 401-416 | GDQYEQADGFPTAQQG | SEQ ID NO: 241 |

In another embodiment, in all the secretory signal peptide sequences, a stretch of four amino acids (LEKR) was added for the efficient Kex2 processing of pre-protein provided in the Table 4.

TABLE 4
Modified Signal Peptides used

| | Modified Signal | | | |
|---|---|---|---|---|
| Sr. | Peptide | SEQ ID | | Length |
| No. | (Source) | NO | Amino Acid Sequence | (a.a.) |
| 1. | FAK - Alpha-factor | SEQ ID | MRFPSIFTAVLFAASSALAAPVN | 85 |
| | (S. cerevisiae) | NO: 228 | TTTEDETAQIPAEAVIGYSDLEG | |
| | | | DFDVAVLPFSNSTNNGLLFINTT | |
| | | | IASIAAKEEGVSLEKR | |
| 2. | FAKS - Alpha- | SEQ ID | MRFPSIFTAVLFAASSALAAPVN | 89 |
| | factor full | NO: 229 | TTTEDETAQIPAEAVIGYSDLEG | |
| | (S. cerevisiae) | | DFDVAVLPFSNSTNNGLLFINTT | |
| | | | IASIAAKEEGVSLEKREAEA | |
| 3. | AT Alpha-factor T | SEQ ID | MRFPSIFTAVLFAASSALALEKR | 23 |
| | (S. cerevisiae) | NO: 230 | | |
| 4. | AA Alpha-amylase | SEQ ID | MVAWWSLFLYGLQVAAPALALEK | 24 |
| | (Aspergillus niger) | NO: 231 | R | |
| 5. | GA Glucoamylase | SEQ ID | MSFRSLLALSGLVCSGLALEKR | 22 |
| | (Aspergillus | NO: 232 | | |
| | awamori) | | | |
| 6. | IN Inulinase | SEQ ID | MKLAYSLLLPLAGVSALEKR | 20 |
| | (Kluyveromyces | NO: 233 | | |
| | maxianus) | | | |
| 7. | IV Invertase | SEQ ID | MLLQAFLFLLAGFAAKISALEKR | 23 |
| | (S. cerevisiae) | NO: 234 | | |
| 8. | KP Killer protein | SEQ ID | MTKPTQVLVRSVSILFFITLLHL | 30 |
| | (S. cerevisiae) | NO: 235 | VVALEKR | |
| 9. | LZ Lysozyme | SEQ ID | MLGKNDPMCLVLVLLGLTALLGI | 30 |
| | (Gallus gallus) | NO: 236 | CQGLEKR | |
| 10. | SA Serum albumin | SEQ ID | MKWVTFISLLFLFSSAYSLEKR | 22 |
| | (Homo sapiens) | NO: 237 | | |

In another embodiment, the modified nucleic acid is cloned in an expression vector.

In another embodiment, the expression vector is configured for secretory or intracellular expression of recombinant FTases from genus Aspergillus.

In yet another embodiment, the expression vector is selected from a group comprising pPICZαA, pPICZαB, pPICZαC, pGAMZαA, pGAMZαB, pGAMZαC, pPIC3, pPIC3.5, pPIC3.5K, PAO815, pPIC9, pPIC9K, pHIL-D2 and pHIL-S1.

In another embodiment, the expression of the modified FTase gene fused to a signal peptide is preferably driven by a constitutive or inducible promoter.

In another embodiment, the nucleic acid to be expressed in operably linked to the promoter.

In another embodiment, the constitutive or inducible promoter is selected from a group listed in Table 5 below.

TABLE 5
List of promoters used

| Sr. | Promoter | Gene | | | Expression |
|---|---|---|---|---|---|
| No. | Type | Name | Gene Product | Inducer | Level |
| 1 | Inducible | AOX1 | Alcohol oxidase 1 | Methanol | Strong |
| 2 | Inducible | ADH3 | Alcohol dehydrogenase | Ethanol | Strong |
| 3 | Inducible | DAS | Dihyroxyacetone phosphate | Methanol | Strong |
| 4 | Inducible | FLD1 | Formaldehyde dehydrogenase | Methanol/ | Strong |
| | | | | Methylamine | |
| 5 | Inducible | LRA3 | L-rhamnonate dehydratase | Rhamnose | 75% of |
| | | | | | pGAP |
| 6 | Inducible | THI11 | Thiamine Biosynthesis | Repressed by | 70% of |
| | | | Protein | Thiamine | pGAP |
| 7 | Constitutive | GAP | Glyceraldehyde 3- | — | strong |
| | | | phosphatedehydrogenate | | |
| 8 | Constitutive | YPT1 | GPTase involved in sectetion | — | weak |
| 9 | Constitutive | TEF1 | Translation elongation factor | — | strong |
| | | | 1 alpha | | |
| 10 | Constitutive | GCW14 | Glycosylphosphatidylinositol | — | strong |
| 11 | Constitutive | PGK1 | Phosphoglycerate kinase | — | 10% of |
| | | | | | pGAP |

In another embodiment, the promoter is an AOX1 promoter, which is induced by methanol and repressed by glucose.

In an embodiment, the expression vector containing the modified gene of interest (FTase gene fused to a nucleic acid encoding signal peptide) is transformed in an appropriate host.

In an embodiment, the method of producing a recombinant host cell capable of expressing modified FTase of Aspergillus sp., process comprising the steps of:
a. synthesizing a modified nucleic acid molecule from Aspergillus sp. as defined in claim 6;
b. constructing a vector harboring the modified nucleic acid molecule; and
c. transforming a host cell with the vector of step (b) to obtain a recombinant host cell.

In an embodiment, the codon optimized gene for β-fructofuranosidase and/or fructosyltransferase has been modified for expression in a heterologous host cell.

In an embodiment, the heterologous recombinant host cell being a prokaryotic host chosen from a group comprising Escherichia coli, Bacillus subtilis, Pseudomonas putida, Corynebacterium glutamicum and the like.

In another embodiment, the heterologous recombinant host cell being a eukaryotic host. The eukaryotic host cell can be chosen from a group comprising Saccharomyces cerevisiae, Pichia pastoris and Hansenula polymorpha and the like.

In another embodiment, the expression vector containing the gene of interest is transformed in yeast cells.

In another embodiment, the yeast cell is a Pichia pastoris.

In yet another embodiment, the Pichia Pastoris host cell is a mut+, mut S or mut-strains. Mut+ represents methanol utilization plus phenotype.

In yet another embodiment, the Pichia Pastoris host cell strain is selected from a group comprising KM71H, KM71, SMD1168H, SMD1168, GS115, X33.

In another embodiment, the invention provides FTase pre-cursor peptides, wherein FTases of genus Aspergillus is fused to one or more signal peptide.

In an important embodiment are provided the mutants and processes of generating mutants of FTases of genus Aspergillus. Mutations can be single, double or triple point mutations as well as deletion mutants and also a combination of these mutations.

In another embodiment, are provided nucleic acid sequences of the modified (3-fructofuranosidase from Aspergillus niger set forth in SEQ ID NO. 9, 11, 13, 15, 17, 19, 21, 23, 25, 27, 29, 31, 33, 35, 37, 39, 41, 43, 45, 47, 49, 51, 53, 55, 57, 59, 61, 63, 65.

In another embodiment, are provided peptide sequences of the modified (3-fructofuranosidase from Aspergillus niger set forth in SEQ ID NO.: 10, 12, 14, 16, 18, 20, 22, 24, 26, 28, 30, 32, 34, 36, 38, 40, 42, 44, 46, 48, 50, 52, 54, 56, 58, 60, 62, 64, 66, 67-218.

In another embodiment, the signal peptide is selected from a group comprising Alpha factor full of S. cerevisiae (FAK) set forth in SEQ ID NO: 219, Alpha-factor full of S. cerevisiae (FAKS) set forth in SEQ ID NO: 229, Alpha factor_T of S. cerevisiae (AT) set forth in SEQ ID NO: 220, Alpha-amylase of Aspergillus niger (AA) set forth in SEQ ID NO: 221, Glucoamylase of Aspergillus awamori (GA) set forth in SEQ ID NO: 222, Inulinase of Kluyveromyces maxianus (IN) set forth in SEQ ID NO: 223, Invertase of S. cerevisiae (IV) set forth in SEQ ID NO: 224, Killer protein of S. cerevisiae (KP) set forth in SEQ ID NO: 225, Lysozyme of Gallus gallus (LZ) set forth in SEQ ID NO: 226, Serum albumin of Homo sapiens (SA) set forth in SEQ ID NO: 227, and variants thereof.

In yet another embodiment, the mutants have been produced by conventional genetic engineering techniques as well as by utilizing bio-informatics based tools. Point mutations were predicted and introduced into the codon-optimized β-fructofuranosidase (fopA) gene of Aspergillus niger and/or ft gene of Aspergillus japonicus. Approaches such as conventional genetic engineering like PCR etc., bioinformatics tools utilizing molecular dynamics simulations using the software GROMACS as well as introduction of codon bias have been used in the present invention.

In one embodiment are provided Fructosyltransferase mutants which are presented as below:

### Point Mutants:

V44L, T155R, T293W, R286V, T459R, R199K, F182P

### Deletion Mutants:

32-654 aa, 32-194 aa, 1-194 aa

In an embodiment, the process for the production of recombinant FTases from genus Aspergillus is provided.

Aspects of the present invention relate to fermentation of recombinant Pichia pastoris cells containing modified/mutated recombinant FTase gene. After completion of the fermentation, the fermentation broth is subjected to centrifugation and filtered using microfiltration and the recombinant enzyme is separated. The recovered recombinant enzyme is concentrated using Tangential Flow Ultra-filtration or evaporation and finally the concentrated enzyme is formulated.

In one embodiment, process for expressing modified FTase of Aspergillus sp. as, comprising:
a. culturing recombinant host cells capable of expressing FTase of Aspergillus sp. in a suitable fermentation medium to obtain a fermentation broth;
b. harvesting supernatant from the fermentation broth, wherein the supernatant contains recombinant FTase; and
c. purifying recombinant FTases.

In another embodiment, the fermentation medium is basal salt medium as described in Table 6 below:

TABLE 6
Composition of basal salt medium

| Component | Concentration | |
|---|---|---|
| Calcium Sulphate | 1.4 | gm/L |
| Potassium Sulphate | 18.6 | gm/L |
| Magnesium Sulphate•7H₂O | 16.4 | gm/L |
| Glycerol | 25 | gm/L |
| Potassium Di hydrogen Phosphate | 5 | gm/L |
| Ammonium Sulphate | 5 | mL |
| Sodium Citrate Di Hydrate | 5 | gm/L |
| PTM2 | 4 | mL |
| Biotin | 4 | mL |

In yet another embodiment, the supernatant from the fermentation broth is harvested using centrifugation.

In one embodiment, the percentage of inoculum or starter culture to initiate the fermenter culture is in the range of 2.0% to 15.0% (v/v).

In another embodiment, the pH of the fermentation medium is maintained in the range of 4.0 to 7.5 as the secreted enzyme undergoes proper folding and is biologically active at this pH range.

In yet another embodiment, the temperature of the fermentation process is in the range of 15° C. to 40° C.

In another embodiment, the time for fermentation process is in the range of 50-150 hrs.

In a further, embodiment, the fermentation broth is centrifuged at a speed in the range from 2000×g to 15000×g using continuous online centrifugation.

The supernatant obtained after centrifugation is subjected to microfiltration and purified to recover biologically active recombinant/mutant FTase.

In one embodiment, the supernatant obtained after centrifugation is concentrated using a Tangential Flow Filtration based Ultra filtration System.

The cut-off size of the membranes used in Tangential Flow Filtration (TFF) systems that may be used to remove impurities and to concentrate the collected culture supernatant may range between 5 to 100 kDa.

In another embodiment, no centrifugation is required for the process due to the high yield and purity of the secreted enzyme.

The FTase concentration obtained in this invention is found to be in the range of or more than 2-5 gm/L and the purity is high, say 85% or more.

In an embodiment, process to identify mutations of amino acids across a FTase protein chain including the active site, wherein the steps comprising:
a. Simulating the apo structure of FTase enzyme using a bioinformatics tool;
b. Clustering the trajectory based on variation of the root mean squared deviation (RMSD) to identify unique conformations;
c. Identifying the unique conformations that the FTase enzyme adopted during the course of the simulation residues for mutation;
d. Analyzing the identified unique conformations of step (c) for pairwise interactions that each amino acid made with another amino acid;
e. Quantifying the pairwise interactions of the amino acids as an indicative interaction strength by providing appropriate weights for the van der Waals and the hydrogen bonds between the pair of residues;
f. Generating 19 mutations of each amino acid that was found to have larger standard deviation in the interaction strengths through the course of the simulation; and
g. Identifying and selecting stabilizing mutations with similar or better stability as per the −ΔΔG values.

### EXAMPLES

The following examples particularly describe the manner in which the invention is to be performed. But the embodiments disclosed herein do not limit the scope of the invention in any manner.

### Example 1: Modified Nucleic Acids for Expression of Recombinant β-Fructofuranosidase of Aspergillus niger in Pichia pastoris

The native cDNA of β-fructofuranosidase (SEQ ID NO. 5) was codon optimized for maximizing expression in Pichia pastoris. The codon optimized nucleotide sequence of β-fructofuranosidase represented by (SEQ ID NO.: 6) is further modified by inducing mutations selected from one or more point mutations as defined in Table 7 or deletion mutations defined in 8 or their combination thereof were induced in the codon optimized nucleotide sequence. The amino acid and nucleotide sequences of modified β-fructofuranosidase is represented by SEQ ID NO.: 9-218.

TABLE 7
A brief of the mutations in FTase proteins

| | | | | | | | | | | | |
|---|---|---|---|---|---|---|---|---|---|---|---|
| N32R | R166Y | H240F | H43Q | N32K | T155W | R286I | V382L | Q406R | A371E | T293Q | F118Y |
| H43Y | R166N | F259W | H43F | H43A | T155Y | L322Y | F568Y | T293F | A371M | T293V | L78R |
| H43V | F182P | R286V | H43S | H43N | T155M | I324Q | Q586K | T293H | E405W | T293Y | L78W |
| H43L | F182R | V343F | P131R | H43K | R166L | V343Q | Q586Y | T293I | N290L | A188H | N290K |
| H43R | F182A | V343L | T155L | V127I | R166Q | V343K | Q586V | T293K | N290M | A188K | N290Q |
| H43T | F182D | A381R | T155V | V127M | H180Y | V343Y | Q586W | T293L | N290R | D191A | Q327C |
| H43M | F182V | A381L | R166K | P131H | H180R | V343I | A188R | T293R | Q327A | D191N | Q327I |
| H43I | F182T | L440R | R166H | P131F | F182M | A381V | D191K | T293S | Q327D | D191W | Q327K |
| V125F | F182L | Q586R | F182E | P131Q | F182H | A381K | D191R | T293W | Q327E | A371L | Q327V |
| V127A | F182W | N32Q | A381I | T132L | F182S | A381P | Q327G | A188F | Q327F | A371V | Q406K |
| P131Y | F182N | N32V | Q586F | T132M | Y232W | A381Q | Q327H | A188Q | Q327R | E405F | Q406M |
| P131W | F182K | H43W | N32D | T132V | H240R | A381T | Q327L | D191E | Q406L | E405G | S329N |
| T155R | F182Q | H43E | N32I | T132I | H240K | A381Y | Q327M | D191F | T293M | E405L | S329T |
| T155K | R196K | H43D | N32Y | T155I | F259Y | V382I | Q327N | D191Q | T293N | E405N | Y404W |
| V44L | K199R | R459T | N654K | L44V | R199K | T459R | K654N | | | | |

TABLE 8
A brief of deletion mutation

| | | |
|---|---|---|
| 32-654 aa, | 32-194 aa, | 1-194 aa |

An expression cassette encoding the β-fructofuranosidase was modified for maximizing expression in Pichia pastoris. The modified open reading frame contains the modified nucleotide sequence encoding β-fructofuranosidase fused to a signal peptide. The nucleic acids have been designed such that the encoded signal peptides contain an additional stretch of four amino acids (LEKR) for the efficient Kex2 processing of precursor peptide.

The preferred codons for expression in Pichia pastoris were used in place of rare codons. The nucleotide sequence of the modified open reading frames encoding for β-fructofuranosidase were fused with modified signal peptides. Recombinant pre-cursor proteins were obtained by translating the gene encoding for β-fructofuranosidase of Aspergillus niger fused with signal peptides. The signal peptides were cleaved off during post-translational modifications inside the Pichia host cells and the mature recombinant β-fructofuranosidase was released into the medium.

### Example 2: Development of Mutants

### Example 2(a): Bio-Informatics Approach

The present inventors sought to identify mutations of amino acids across the protein chain including the active site that can result in greater stabilization of the protein AnFT i.e., enzyme FT from Aspergillus niger. In order to identify such amino acids, the apo structure of AnFT was subjected to 25 ns molecular dynamics simulations using the software GROMACS.

Subsequent to the simulation, the trajectory was clustered based on the variation of the root mean squared deviation (RMSD) of the CA atoms to identify unique conformations that the protein adopted during the course of the simulation. Representative structures of the clusters (cluster centres) were chosen.

These structures were analysed for the interactions that each amino acid made with another amino acid. The pairwise interactions of the amino acids were quantified as an indicative interaction strength by providing appropriate weights for the van der Waals and the hydrogen bonds between the pair of residues.

Strength of interaction between amino acids i and j, Sij=Wt/rij

Where, Wₜ is the weight for a given type of interaction and rij is the distance between atoms of the residues i and j. The strength of the interaction computed between two residues is the algebraic sum of the strengths of interactions between atoms of the two residues.

This yielded an N×N matrix for a protein of N residues with each element representing the interaction strength and 0 representing no interaction. Sum of each row in the matrix simply yields the overall interaction strength of a given amino acid.
e.g., total interaction strength of residue 100=sum (interaction strength of residue 100 with every other residue)

A radius of 5∈ was chosen as a cut off to analyse and quantify the interactions. No interaction was assumed between successive amino acids so that the interaction strength was a true reflection of the stabilization through tertiary structure. Greater interaction strength points to greater stabilization of the amino acid and hence that of the protein.

The ensemble of AnFT structures resulting from the molecular dynamics simulations followed by clustering comprised of 5-6 different conformations (cluster centres). For each of the conformation, the interaction strength matrix was generated and the individual interaction strength of each amino acid was computed as described above.

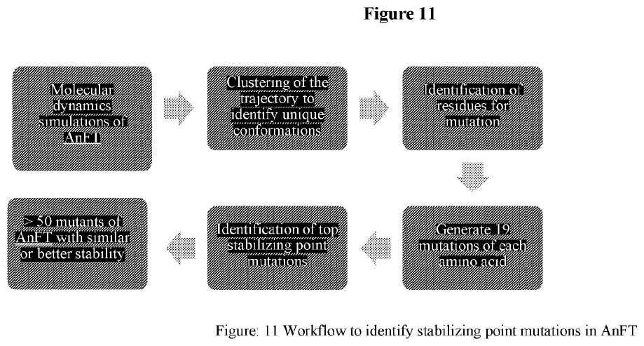

The variation of the interaction strengths of each amino acid across the ensemble was studied using the statistical parameter of standard deviation. A greater standard deviation in the interactions among the various conformations indicated a flexible residue while a lower standard deviation indicated a well networked residue. The flexible residues were targeted to generate mutants. An overview of the scheme for identification of the most stable sites for mutations is illustrated in FIG. 11.

Further, each such amino acid that was found to have larger standard deviation in the interaction strengths through the course of the simulation was mutated into all other 19 natural amino acids occurring in proteins.

Furthermore, energies of stabilization were computed for the mutated amino acids with respect to the wildtype and they were represented by the parameter ΔΔG indicating the difference between the ΔG for the wild type and the stabilized mutant. This analysis was carried out using the commercial software ICM from MolSoft. The ΔΔG (kcal/mol) values for the mutation of each amino acid into 19 others were computed and those mutations which showed no further or better stabilization were selected. The list of mutations so selected are given in the Table 9 below along with the ΔΔG (kcal/mol) values for each mutation.

TABLE 9

| | −ΔΔG | | −ΔΔG | | −ΔΔG |
|---|---|---|---|---|---|
| Mutation | (kcal/mol) | Mutation | (kcal/mol) | Mutation | (kcal/mol) |
| N32R | −0.89 | Q586R | −1.10 | F182S | 0.16 |
| H43Y | −0.75 | N32Q | −0.23 | Y232W | 0.14 |
| H43V | −0.74 | N32V | −0.21 | H240R | −0.03 |
| H43L | −0.71 | H43W | −0.35 | H240K | 0.12 |
| H43R | −0.61 | H43E | −0.34 | F259Y | −0.02 |
| H43T | −0.54 | H43D | −0.33 | R286I | 0.13 |
| H43M | −0.53 | H43Q | −0.28 | L322Y | −0.13 |
| H43I | −0.46 | H43F | −0.27 | I324Q | 0.08 |
| V125F | −0.90 | H43S | −0.20 | V343Q | −0.06 |
| V127A | −0.77 | P131R | −0.25 | V343K | 0.08 |
| P131Y | −0.64 | T155L | −0.30 | V343Y | 0.11 |
| P131W | −0.45 | T155V | −0.20 | A381Q | −0.03 |
| T155R | −1.68 | R166K | −0.35 | A381T | 0.01 |
| T155K | −0.43 | R166H | −0.24 | V382L | 0.12 |
| R166Y | −0.58 | F182E | −0.30 | F568Y | −0.16 |
| R166N | −0.56 | A381I | −0.36 | Q586K | −0.11 |
| F182P | −1.31 | Q586F | −0.20 | T155M | 0.16 |
| F182R | −1.24 | N32D | 0.02 | R166L | −0.15 |
| F182A | −0.98 | N32I | 0.05 | R166Q | −0.15 |
| F182D | −0.93 | N32Y | 0.06 | H180Y | −0.14 |
| F182V | −0.91 | N32K | 0.11 | H180R | 0.16 |
| F182T | −0.88 | H43A | −0.15 | F182M | −0.10 |
| F182L | −0.48 | H43N | −0.14 | F182H | 0.11 |
| F182W | −0.47 | H43K | 0.00 | V343I | 0.13 |
| F182N | −0.47 | V127I | 0.04 | A381V | −0.20 |
| F182K | −0.46 | V127M | 0.16 | A381K | −0.11 |
| F182Q | −0.43 | P131H | −0.15 | A381P | −0.05 |
| R196K | −0.47 | P131F | −0.12 | A381Y | 0.15 |
| H240F | −0.41 | P131Q | 0.17 | V382I | 0.07 |
| F259W | −0.49 | T132L | −0.19 | Q586Y | −0.09 |
| R286V | −1.51 | T132M | −0.09 | Q586V | −0.08 |
| V343F | −0.81 | T132V | 0.02 | Q586W | 0.00 |
| V343L | −0.52 | T132I | 0.07 | A381L | −0.42 |
| A381R | −0.50 | T155I | −0.11 | L440R | −0.53 |
| T155Y | 0.14 | T155W | −0.08 | | |

Similar exercise as described above was carried out on the kestose bound AnFT structure to analyse the strengths of interactions of the amino acids in the kestose binding site and the effect of their mutations on the stability of the protein. The results of that analysis are shown in the below Table 10.

TABLE 10

| | −ΔΔG | | −ΔΔG | | −ΔΔG |
|---|---|---|---|---|---|
| Mutant | (kcal/mol) | Mutant | (kcal/mol) | Mutant | (kcal/mol) |
| A188R | −1.31 | T293K | −1.25 | E405W | −0.53 |
| D191K | −1.04 | T293L | −1.43 | N290L | −0.55 |
| D191R | −1.10 | T293R | −2.52 | N290M | −0.51 |
| Q327G | −1.19 | T293S | −1.51 | N290R | −0.75 |
| Q327H | −1.06 | T293W | −3.51 | Q327A | −0.54 |
| Q327L | −1.43 | A188F | −0.76 | Q327D | −0.71 |
| Q327M | −1.31 | A188Q | −0.66 | Q327E | −0.94 |
| Q327N | −1.50 | D191E | −0.62 | Q327F | −0.51 |
| Q406R | −1.14 | D191F | −0.65 | Q327R | −0.92 |
| T293F | −1.89 | D191Q | −0.96 | Q406L | −0.73 |
| T293H | −1.75 | A371E | −0.66 | T293M | −0.88 |
| T293I | −0.62 | A371M | −0.66 | T293N | −0.66 |
| A371V | −0.06 | Q327V | −0.19 | T293Q | −0.81 |
| E405F | −0.20 | Q406K | −0.33 | T293V | −0.67 |
| E405G | −0.35 | Q406M | −0.18 | T293Y | −0.71 |
| E405L | −0.16 | S329N | −0.23 | A188H | −0.45 |
| E405N | −0.16 | S329T | −0.37 | A188K | −0.31 |
| F118Y | 0.00 | Y404W | −0.07 | D191A | −0.21 |
| L78R | −0.16 | N290Q | −0.47 | D191N | −0.12 |
| L78W | −0.24 | Q327C | −0.01 | D191W | −0.33 |
| N290K | −0.27 | Q327I | −0.24 | A371L | −0.35 |
| Q327K | −0.02 | | | | |

As described above, utilizing a combination of molecular dynamics simulations, trajectory analyses, interactions strengths of individual amino acids and the characterization of the mutants for their stabilizing influence on the protein, a minimum of 172 sites for point mutation were identified. Further, the suitable sites for deletion were also identified based on the structural studies in the prior art on the apo structure of the FTase.

### Example 2(b): Genetic Engineering Approach

Based on bioinformatics approaches the alternative forms of functional domains for the FTase protein were conceived and cloned to further test their efficacy for the process. For cloning work, the primers were designed to PCR amplify a particular stretch of DNA from the recombinant plasmid used as a template having codon optimized full length FTase gene. Thus, the amplified PCR products contained the required deletion and retained the desired stretch from the gene. These PCR products were cloned in the respective expression vectors with compatible strategies, for example cloned as XhoI and SacII fragments at the corresponding sites in the MCS of vector pPICZαA.

The point mutations were introduced in the sequence of FTase gene through primers carrying the mutated nucleotides. The PCR products generated through these primers contained the mutated sequence at the corresponding site(s). These PCR products now carrying the point mutations were then cloned according to the compatible strategy in the desired expression vector.

### Example 3: Development of Recombinant Host Cells by Transformation with Recombinant Plasmids

The vector used in the process was pPICZαA. The vectors contained the modified open reading frames and an inducible promoter, AOX1. The modified sequence encoding for the recombinant protein was cloned into the pPICZαA vector.

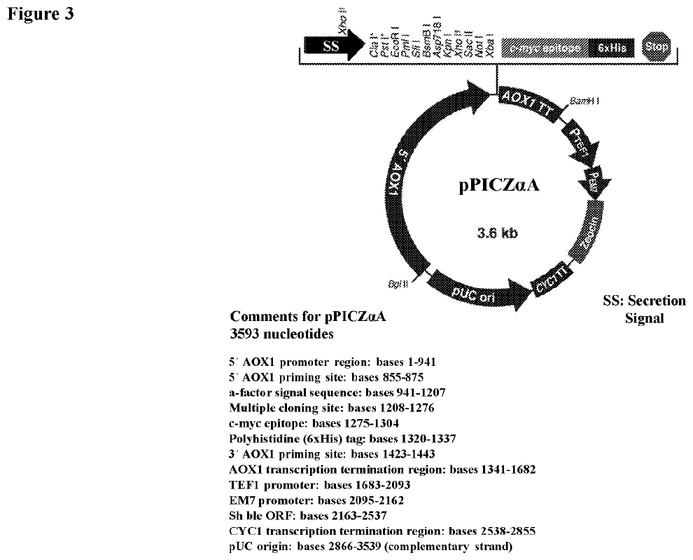

The modified nucleic acid encoding respective FTase gene was cloned between XhoI SacII restriction sites present in the MCS of pPICZαA vector to bring signal sequence Alpha-factor of S. cerevisiae (FAK) in frame to create expression cassette using regular molecular biology procedures. The vector map for pPICZαA is represented in FIG. 3.

The putative recombinant plasmids were selected on low salt-LB media containing 25 μg/ml Zeocin and screened by XhoI/SacII restriction digestion analysis.

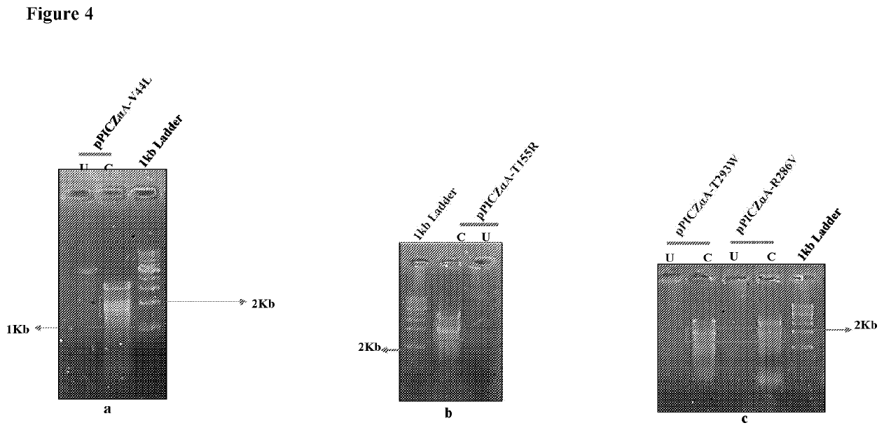

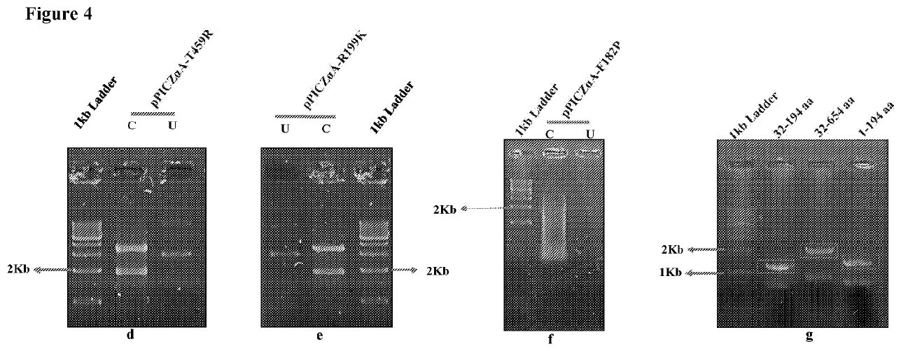

The recombinant plasmid pPICZαA-FTase was confirmed by XhoI SacII restriction digestion analysis which resulted in release of 1980 bp fragment. The results of the restriction digestion analysis are depicted in FIG. 4.

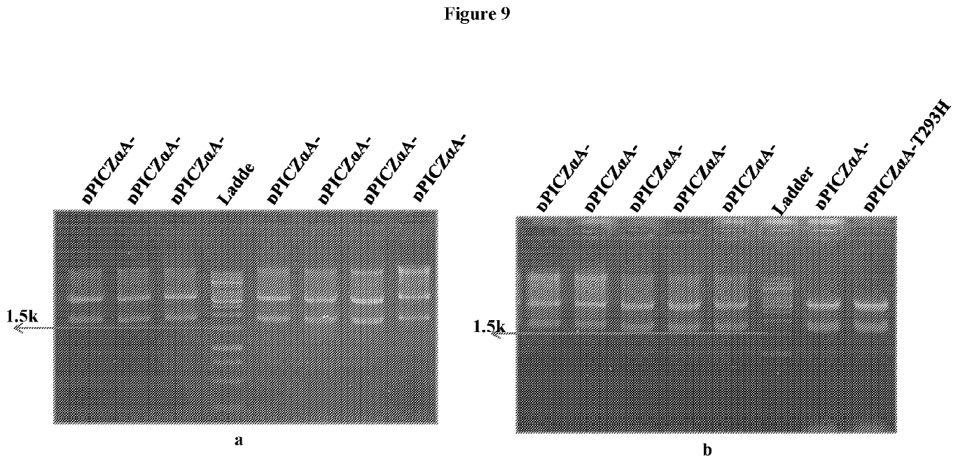

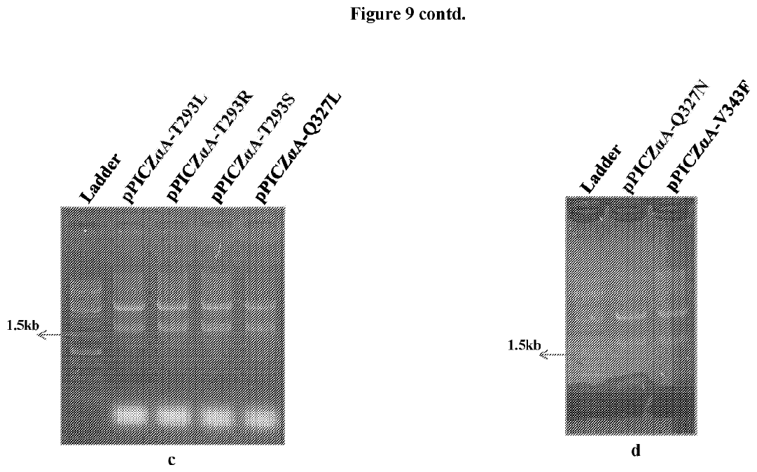

FIG. 9 depicts the results of the restriction digestion analysis performed on the recombinant plasmid pPICZαA containing (a): N32R, H43Y, H43V, H43L, V125F, V127A, P131Y; (b): F182R, F182A, F182D, F182V, F182T, T293F, T293H; (c): T293R, T293L, T293S, Q327L; (d): Q327N, V343F Thereafter, Pichia pastoris cells were electroporated with linearized recombinant pPICZαA-FTase DNA. The Pichia integrants were selected on yeast extract peptone dextrose sorbitol agar (YPDSA) containing 100 μg/ml Zeocin.

The integration was screened with colony PCR (cPCR). For cPCR, a template from each of the Pichia integrants was generated by the alkali lysis method.

The Pichia integrants were grown for 48h in BMD1 media and further induced first with BMM2 and then successively with BMM10 media which provided final concentration of 0.5% methanol in the culture medium. At the end of 96 hrs induction period, culture supernatants from different clones were harvested. Total protein from each of the harvested supernatants was precipitated with 20% TCA and analyzed on SDS-PAGE.

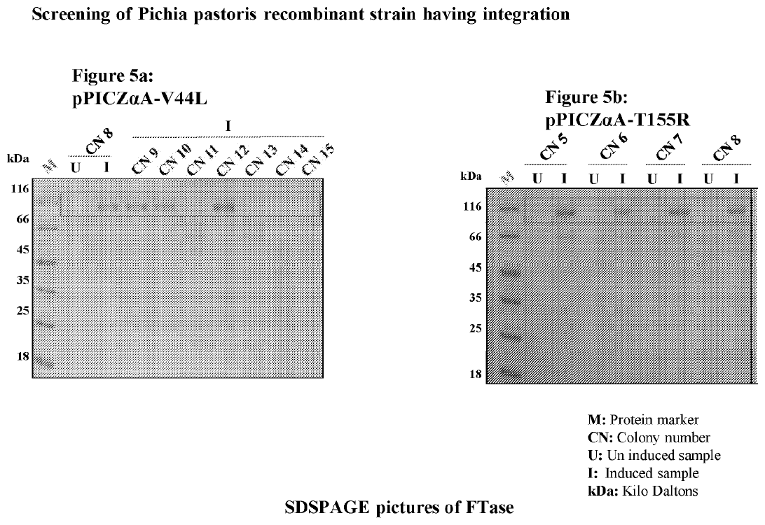

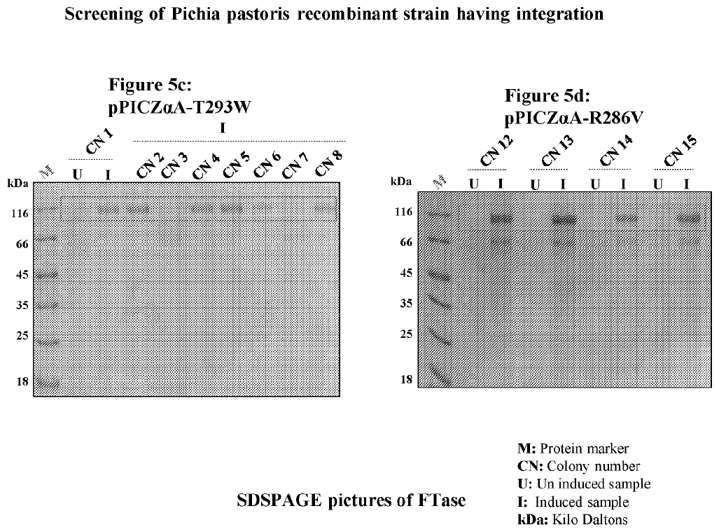

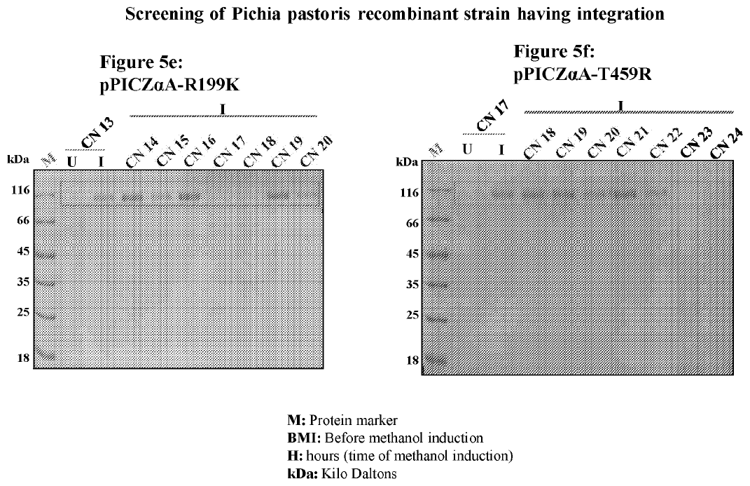

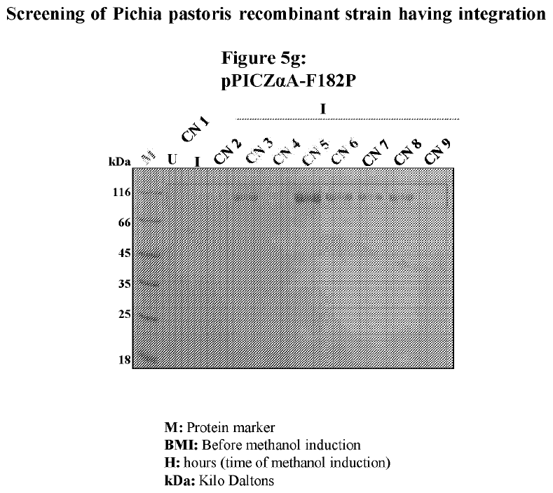

Upon induction FTase protein bands were seen at the size of approximately 110 kDa as depicted in FIG. 5.

The calculated molecular weight was about 70.85 kDa. The increase in molecular weight may have been contributed by glycosylation.

### Example 4: Fermentation of Recombinant Pichia pastoris Expressing Mutant FTases of Aspergillus

Fermentation of recombinant Pichia pastoris cells containing the modified FTase gene as described in Example 1 was carried out in a 50 L fermenter. Fermentation was carried out in basal salt medium as described herein.

### Preparation of Pre-Seed and Seed Inoculum:

The pre-seed was generated by inoculating from the glycerol stock in 25 mL of sterile YEPG medium and growing at 30° C. in a temperature-controlled orbital shaker overnight. For generating seed, the inoculum was grown in Basal salt medium in baffled shake flasks at 30° C. in a temperature-controlled orbital shaker till OD₆₀₀ of 15-25 was reached.

### Fermentation Process

The entire process of fermentation from the inoculation of fermenter with seed culture to final harvesting took about 130 hrs. Basal salt medium was prepared and sterilized in situ in the fermenter.

The composition of basal salt medium optimized for the fermentation process is provided in Table 6.

Pichia Trace Minerals (PTM) salt solution was prepared with the components mentioned in Table 11. PTM salts were dissolved and made up to 1 L volume and filter sterilized. PTM salt solution was included at the rate of 4 ml per liter of initial media volume after sterilization of the basal salt media.

TABLE 11
PTM trace salts

| Component | Concentration | |
|---|---|---|
| Cupric sulfate•5H₂O | 2.0 | gm/L |
| Sodium iodide | 0.08 | gm/L |
| Manganese sulfate•H₂O | 3.0 | gm/L |
| Sodium molybdate•2H₂O | 0.2 | gm/L |
| Boric Acid | 0.02 | gm/L |
| Cobalt chloride | 0.5 | gm/L |
| Zinc Sulphate | 7.0 | gm/L |
| Ferrous sulfate•7H₂O | 22.0 | gm/L |
| Potassium chloride | 0.37 | gm/L |
| Sulfuric Acid | 1 | mL |
| Ferric chloride | 0.811 | gm/L |
| Nickel chloride | 1.18 | gm/L |
| Magnesium sulfate | 1.23 | gm/L |

### Growth Phase:

The growth phase was initiated by inoculating basal salt medium in 50 L fermenter with 5% seed culture and continued for about 24 hours. The dissolved oxygen (DO) levels were continuously monitored and never allowed to drop below 40%.

After 18h, a DO spike was observed indicating the depletion of carbon source (Glycerol). A glycerol fed-batch was initiated by feeding 50% Glycerol (with 12 ml of PTM salts per liter of feed) for about six hours till the OD₆₀₀ reached 200.

### Induction Phase:

Once sufficient biomass was generated, the induction phase was initiated by discontinuing glycerol feed and starting methanol feed. Methanol (supplemented with 12 ml of PTM salts per liter of feed) was fed at the rate of 0.5 g to 3 g per liter of initial fermentation volume. The DO was maintained at 40% and methanol feed was accordingly adjusted.

The induction of FTase gene was monitored periodically by analyzing culture supernatant by enzyme activity assay. The induction phase was continued for about 100 hours till the OD₆₀₀ reached 600 and wet biomass reached ˜560 grams per liter of culture broth.

The fermentation was stopped after 130 hours and enzyme activity in the fermenter broth was determined at the end of fermentation by DNS method (Miller, 1959). One unit is defined as the amount of enzyme required to release one micromole of reducing sugars (glucose equivalents) from 10% sucrose solution in 100 mM citrate buffer pH 5.5 at 55° C. The total amount of recombinant FTase in the culture broth was estimated by Bradford assay.

### Fermentation Conditions:

The fermentation parameters considered were as given in Table 12. These essential parameters were monitored during the fermentation process.

TABLE 12
Fermentation Parameters

| | | |
|---|---|---|
| Fermentation parameters | Growth phase | Induction phase |

| | | |
|---|---|---|
| Media | Basal Salt Media | Basal Salt Media |
| pH | 5 | 5 |
| Temperature | 30 | 25 |

| | | | | |
|---|---|---|---|---|
| Agitation (tip speed) | 1.2-2.5 | m/Sec | 2.5 | m/Sec |
| Aeration | 0.5-1.5 | vvm | 1.5 | vvm |

| | | |
|---|---|---|
| Dissolved oxygen | Minimum 40% | Minimum 40% |

| | | | | |
|---|---|---|---|---|
| Back pressure | 0.5 | kg/cm² | 0.5 | kg/cm² |

### Example 5: Cell Harvesting and Purification

Harvesting of the enzyme was performed by continuous centrifugation at 8000 RPM. Clear supernatant obtained after centrifugation was subjected to microfiltration using 0.1 microns cut off spiral wound TFF membrane. The filtrate was further subjected to ultrafiltration and diafiltration using 10 kDa cutoff spiral wound TFF membrane and sufficiently concentrated and to reach the desired activity. The enzyme was formulated by including 35-50% of glycerol and food-grade preservatives in the final preparation. The final purity of the enzyme was observed to be 85% as determined by SDS-PAGE analysis.

SDS-PAGE analysis of samples was collected at different time intervals during fermentation of Pichia pastoris strain expressing recombinant FTase enzyme was also performed. SDS-PAGE analysis of recombinant Ftase enzyme after purification was also performed.

FIG. 5 depicts the SDS-PAGE analysis for screening protein expression in Pichia pastoris recombinant strains having integration (a): pPICZαA-V44L; (b): pPICZαA-T155R; (c): pPICZαA-T293W; (d): pPICZαA-R286V; ©: pPICZαA-R199K; (f): pPICZαA-T459R; (g): pPICZαA-F182P.

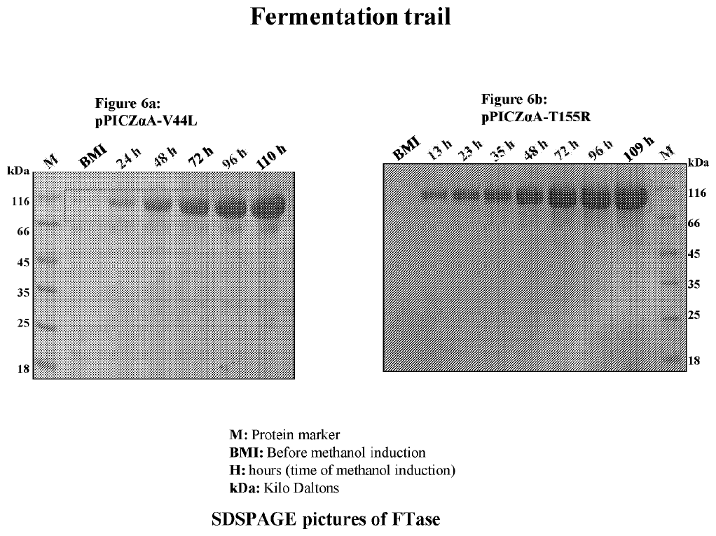

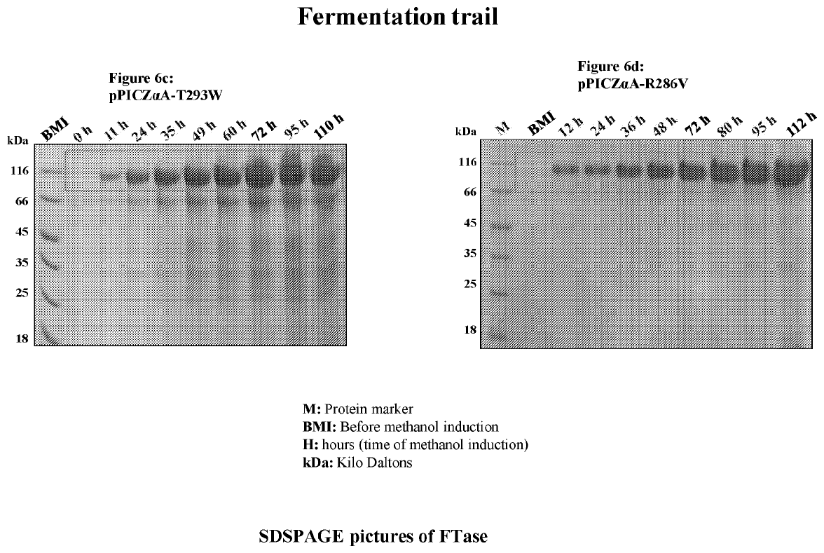

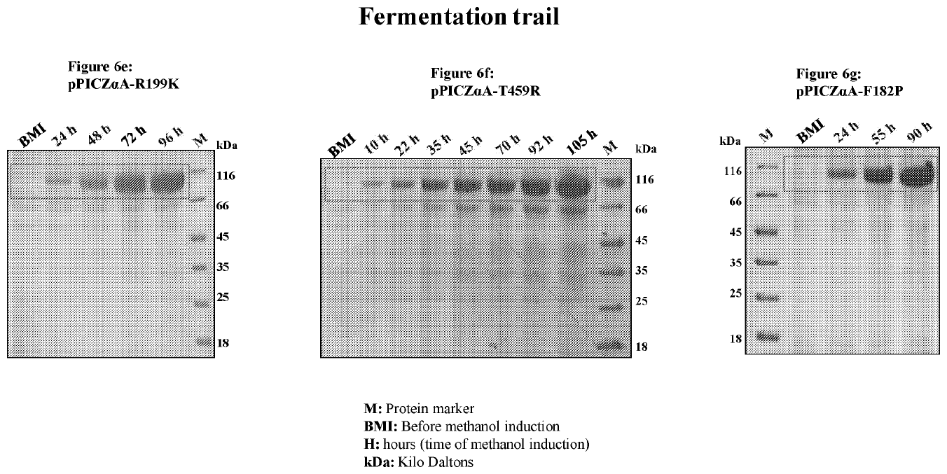

FIG. 6 depicts the SDS-PAGE analysis of samples collected at different time intervals during fermentation trail of various mutant Ftase proteins. (a): pPICZαA-V44L; (b): pPICZαA-T155R; (c): pPICZαA-T293W; (d): pPICZαA-R286V; ©: pPICZαA-R199K; (f): pPICZαA-T459R; (g): pPICZαA-F182P

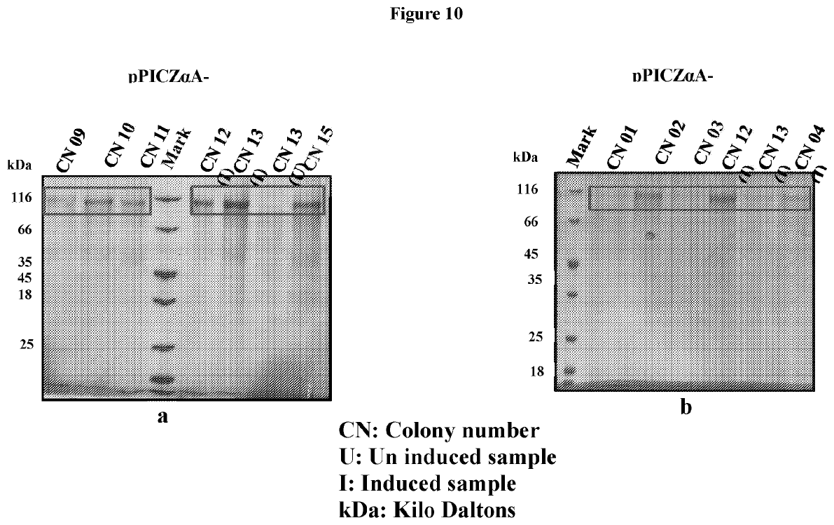

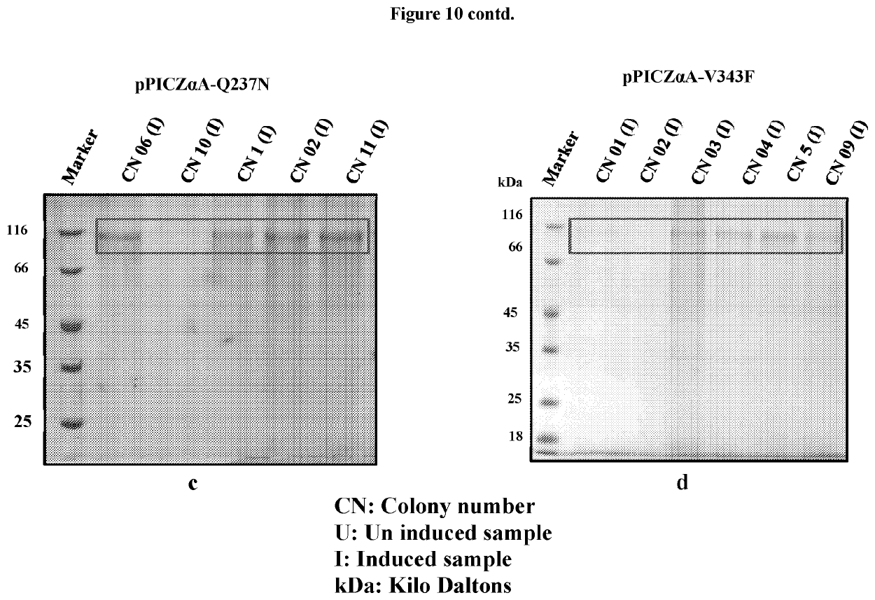

FIG. 10 depicts the SDS-PAGE analysis for screening protein expression in Pichia pastoris recombinant strains having integration (a): pPICZαA-H43Y; (b): pPICZαA-H43L; (c): pPICZαA-Q327N; (d): pPICZαA-V343F

The Ftase concentration was found to be about 2.4 gm/L. In most of the batches, the concentration was 2-5 gm/L. The purity of the recombinant Ftase was observed to be about 85%.

### Example 6: Estimation of Ftase Activity

Studies were conducted to estimate the activity of Ftase. For the estimation studies, the amount of reducing sugar generated due to the action of Ftase enzyme was calculated using DNS (3,5 Dinitrosalicylic acid) method (G. L. Miller, “Use of dinitrosalicylic acid reagent for determination of reducing sugar”, Anal. Chem., 1959, 31, 426-428).

For conducting the enzyme activity assay, 100 Sucrose (dissolved in 100 mM Citrate buffer) was used as the substrate. Ftase was recovered from the fermentation broth and processed through ultra-filtration. The ultra-filtered sample was then diluted 25,000× by serial dilution in 100 mM Citrate buffer and was used. The reaction volume was 2.5 mL. The pH was maintained at 5.5 and the reaction was continued for 15 minutes.

After incubation 3 mL of DNS (3,5 Dinitrosalicylic acid) was added to each reaction mixture and boiled for 10 min, cooled and read absorbance at 540 nm, spectrophotometrically. The OD of glucose at different concentration was measured as shown in Table 13. Thereafter, based on the absorbance measurement after the reaction, the enzyme activity was calculated as shown in Table 14.

TABLE 13
OD measurement of glucose at different concentration

| Glucose(μmol) | OD at 540 nm |
|---|---|
| 0 | 0 |
| 0.055 | 0 |
| 0.55 | 0.018 |
| 1.1 | 0.165 |
| 1.65 | 0.289 |
| 2.2 | 0.452 |
| 2.75 | 0.619 |
| 3.33 | 0.77 |
| 3.85 | 0.891 |
| 4.44 | 1.052 |
| 4.95 | 1.198 |
| 5.5 | 1.338 |

TABLE 14
Estimation of activity of FTase

| Reaction test | Buffer | Substrate | Enzyme | OD | Effective | |
|---|---|---|---|---|---|---|
| tubes | (mL) | (mL) | (mL) | @540 nm | OD | Unit/mL |
| Reagent | 2.5 | — | — | 0.000 | — | — |
| blank | | | | | | |
| Substrate | 0.1 | 2.4 | — | 0.230 | — | — |
| blank | | | | | | |
| Enzyme | 2.4 | — | 0.1 (25,000X | 0.000 | — | — |
| blank | | | diluted) | | | |
| Enzyme | — | 2.4 | 0.1 (25,000X | 0.940 | 0.71 | 51692 |
| Reaction | | | diluted) | | | |

The FTase activity of different clones were estimated after 96 hrs of methanol induction. Supermatant of each clone was subjected to TCA precipitation. The TCA prepped sample was then tested for FTase activity (by DNS method) according to the reference mentioned above. Multiple clones of each construct were tested for FTase activity. The data from representative clones have been presented in Table 15. The FTase activity of the wild type molecule found to be 290 Units/ml was considered as experimental control.

The FTase activity of mutant variants namely but not limited to N32R, H43V, V125F, V127A, P131Y, T155R, F182P, F182R, F182A, F182D, F182V, F182T, R286V, T293F, T293H, T293L, T293S, T293W, H43Y, H43L, Q327N, Q327L and V343F were found to be surprisingly well.

TABLE 15

| Type of | Construct name/ | Activity |
|---|---|---|
| Mutation | clone number | Units/ml |
| Point | pPICZαA-N32R/CN9 | 222 |
| Mutation | pPICZαA-H43Y/CN13 | 533 |
| | pPICZαA-H43V/CN16 | 110 |
| | pPICZαA-H43L/CN12 | 368 |
| | pPICZαA-V125F/CN 1 | 180 |
| | pPICZαA-V127A/CN 11 | 187 |
| | pPICZαA-P131Y/CN19 | 92 |
| | pPICZαA-T155R/CN 5 | 160 |
| | pPICZαA-F182R/CN 3 | 140 |
| | pPICZαA-F182A/CN 1 | 102 |
| | pPICZαA-F182D/CN 3 | 120 |
| | pPICZαA-F182V/CN 12 | 111 |
| | pPICZαA-F182T/CN 1 | 108 |
| | pPICZαA-R286V/CN 13 | 163 |
| | pPICZαA-T293F/CN 22 | 93 |
| | pPICZαA-T293H/CN 23 | 40 |
| | pPICZαA-T293L/CN 9 | 58 |
| | pPICZαA-T293R/CN 8 | 29 |
| | pPICZαA-T293S/CN 7 | 269 |
| | pPICZαA-T293W/CN 5 | 190 |
| | pPICZαA-Q327L/CN 6 | 293 |
| | pPICZαA-Q327N/CN 11 | 354 |
| | pPICZαA-V343F/CN 5 | 371 |
| Deletion mutation | pPICZαA-(32-654 aa)/CN1 | 200 |
| | pPICZαA-(32-194 aa)/CN3 | 67 |
| | pPICZαA-(1-194 aa)/CN1 | 78 |
| Combination of deletion and | pPICZαA-(32-654 aa)H43Y | 210 |
| point mutation | V343F | |

### Example 7: Generation of Fructooligosaccharides (FOS) from Sucrose and Recombinant FTase Enzyme

Studies were conducted to understand the ability of the enzyme in the formation of fructooligosaccharides. A 100 mL solution of 90% (w/v) Sucrose was prepared in 150 mM sodium citrate buffer pH 5.5. To this, 96.7 μL of FTase enzyme having 51692 Unit/ml of activity (equivalent to total of 5000 Units of enzyme), was added.

The reaction was set up in a 250 mL conical flask and incubated at 65° C. and 220 rpm. At regular time intervals, samples were taken and analyzed on Thin Layer Chromatographic (TLC) plates.

Glucose, sucrose, fructose and FOS (containing kestose, nystose and fructofuranosylnystose) were used as standards for the thin layer chromatographic analysis. The mobile phase used was n-Butanol: Glacial acetic acid:Water (4:2:2 v/v) and the developing/staining solution used was urea phosphoric acid.

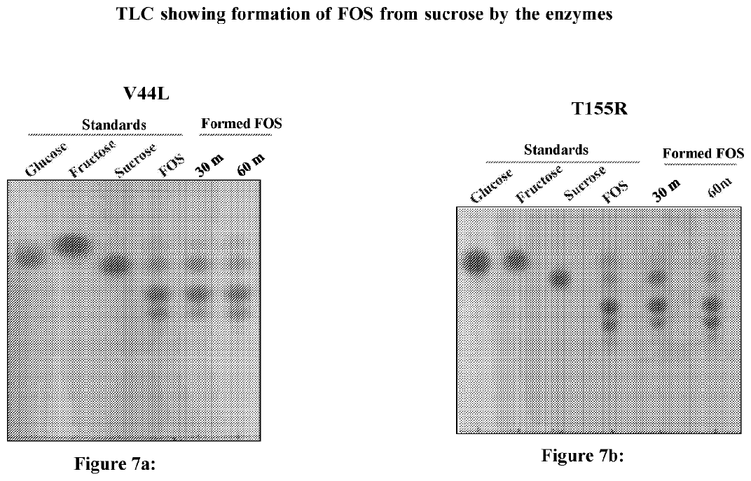

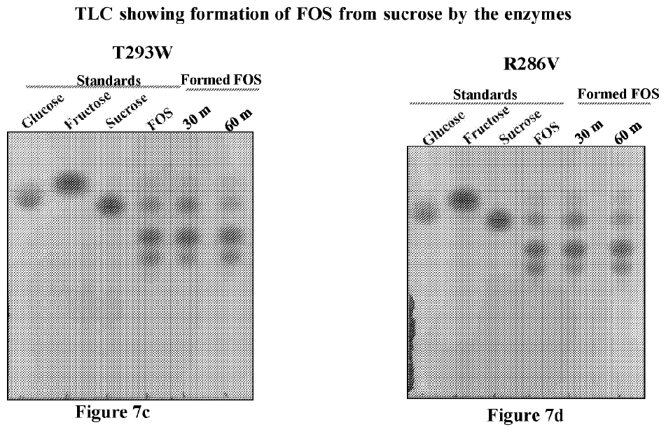

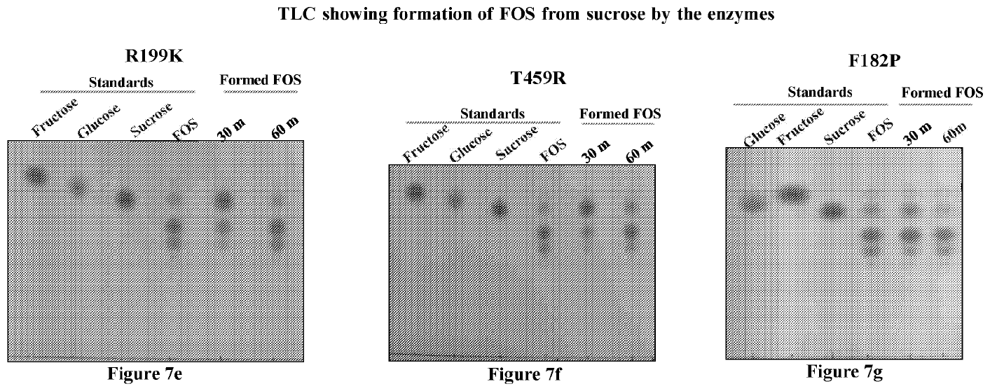

FIG. 7 depicts the TLC analysis done for the generation of fructooligosaccharides (FOS) from sucrose and recombinant FTase enzyme.

The sample was further subjected to High Performance Liquid Chromatography (HPLC) for quantitative estimation of the production of fructooligosaccharides. The HPLC analysis was done using an amine column (Zorbax NH₂ column, Agilent Technologies) having 4.6 (ID)×150 mm (length) and 5 μm (particle size). The standard solutions of glucose, fructose, kestose, nystose, fructofuranosylnystose and sucrose of different concentrations were run for generating standard curves.

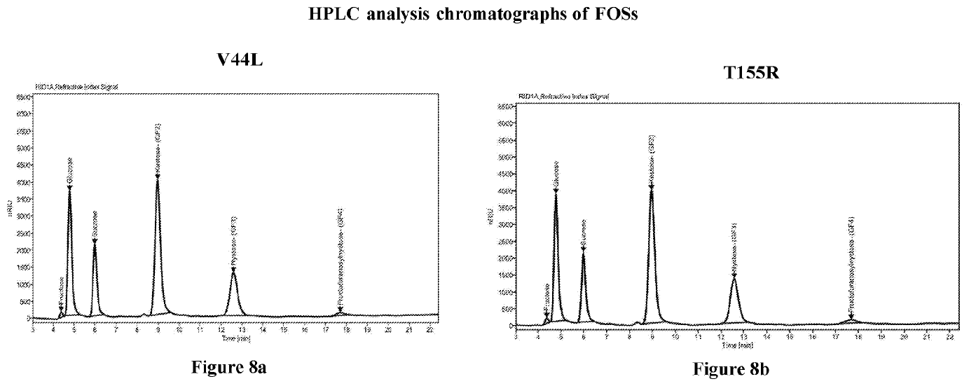

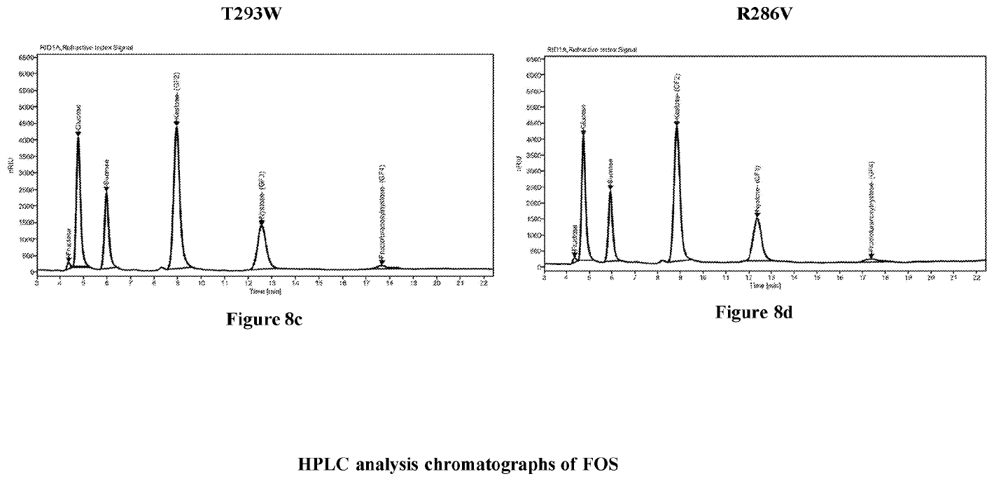

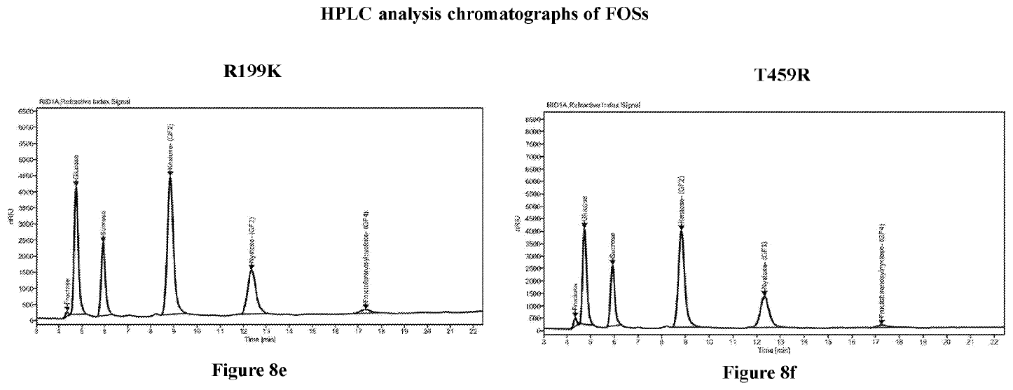

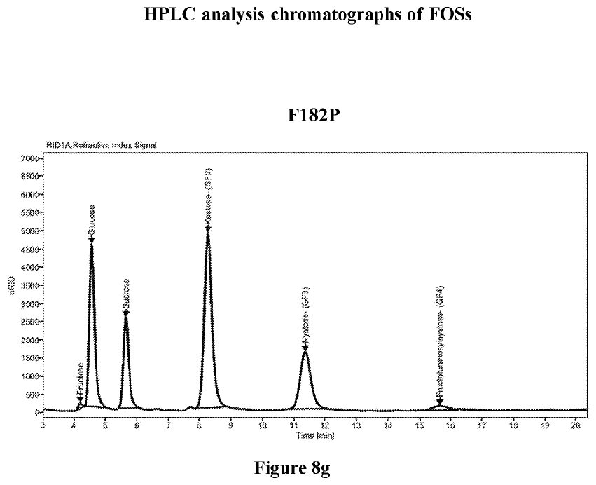

FIG. 8 depicts the HPLC analysis chromatogram of FOS samples.

### Example 8: Characterization of Recombinant FTase of Aspergillus sp.

The harvested FTase of Aspergillus sp. selected from but not limited to fructosyltransferase of Aspergillus japonicus, β-fructofuranosidase of Aspergillus niger was characterized to identify bioactive fragments. It was found that following bioactive fragments of are conserved and accounts for the catalytic activities as illustrated in Table 3 above.

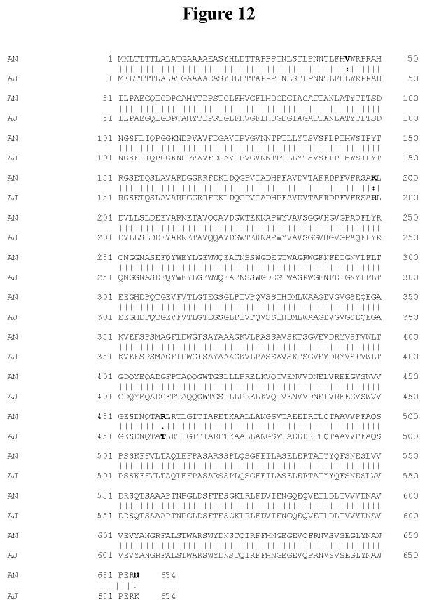

Similar approach as described above was followed for creating mutants of FTase of Aspergillus japonicus. The protein sequence of FTase in A. niger and A. japonicus differ only at four loci out of 654 loci namely, 44, 199, 459 and 654 as illustrated in FIG. 12. In terms of activity, both the enzymes (from genes ft and fopA) were found to be comparative with each other.

### ADVANTAGES

1. The enzymes exhibits high purity after filtration, which eliminates the need for costly chromatographic procedures.
2. The enzymes can be used for obtaining a high yield of fructooligosaccharides.

1. A modified polypeptide, wherein the polypeptide is a FTase of Aspergillus sp. comprising mutations selected from but not limited to single point mutations or multiple point mutations or deletion mutations or a combination thereof.

2. The modified polypeptide as claimed in claim 1, wherein the point mutations are selected from but not limited to N32R, R166Y, H240F, H43Q, N32K, T155W, R286I, V382L, Q406R, A371E, T293Q, F118Y, H43Y, R166N, F259W, H43F, H43A, T155Y, L322Y, F568Y, T293F, A371M, T293V, L78R, H43V, F182P, R286V, H43S, H43N, T155M, I324Q, Q586K, T293H, E405W, T293Y, L78W, H43L, F182R, V343F, P131R, H43K, R166L, V343Q, Q586Y, T293I, N290L, A188H, N290K, H43R, F182A, V343L, T155L, V127I, R166Q, V343K, Q586V, T293K, N290M, A188K, N290Q, H43T, F182D, 381R, T155V, V127M, H180Y, V343Y, Q586W, T293L, N290R, D191A, Q327C, H43M, F182V, A381L, R166K, P131H, H180R, V343I, A188R, T293R, Q327A, D191N, Q327I, H43I, F182T, L440R, R166H, P131F, F182M, A381V, D191K, T293S, Q327D, D191W, Q327K, V125F, F182L, Q586R, F182E, P131Q, F182H, A381K, D191R, T293W, Q327E, A371L, Q327V, V127A, F182W, N32Q, A381I, T132L, F182S, A381P, Q327G, A188F, Q327F, A371V, Q406K, P131Y, F182N, N32V, Q586F, T132M, Y232W, A381Q, Q327H, A188Q, Q327R, E405F, Q406M, P131W, F182K, H43W, N32D, T132V, H240R, A381T, Q327L, D191E, Q406L, E405G, S329N, T155R, F182Q, H43E, N32I, T132I, H240K, A381Y, Q327M, D191F, T293M, E405L, S329T, T155K, R196K, H43D, N32Y, T155I, F259Y, V382I, Q327N, D191Q, T293N, E405N, Y404W, V44L, K199R, R459T, N654K, L44V, R199K, T459R, K654N.

3. The modified polypeptide as claimed in claim 1, wherein the deletion mutations are selected from but not limited to 32-654 aa, 32-194 aa, 1-194 aa.

4. The modified polypeptide as claimed in claim 1, wherein the polypeptide sequences are selected from but not limited to Seq ID NO: 9-218.

5. The modified polypeptide as claimed in claim 4, wherein the polypeptide sequences are selected from Seq ID NO.: 9-66.

6. A polynucleotide comprising a nucleic acid molecule encoding for the modified polypeptide as claimed in claim 1.

7. The polypeptide as claimed in claim 1, wherein the amino acid sequence or nucleotide sequence is further fused to a signal peptide selected from a group comprising FAK, FAKS, AT, AA, GA, IN, IV, KP, LZ and SA or variants thereof.

8. The modified polypeptide as claimed in claim 7, wherein:
a. FAK comprises the amino acid sequence of SEQ ID NO: 219 or variants thereof;
b. FAKS comprises the amino acid sequence of SEQ ID NO: 229 or variants thereof;
c. AT comprises the amino acid sequence of SEQ ID NO: 220 or variants thereof;
d. AA comprises the amino acid sequence of SEQ ID NO: 221 or variants thereof;
e. GA comprises the amino acid sequence of SEQ ID NO: 222 or variants thereof;
f. IN comprises the amino acid sequence of SEQ ID NO: 223 or variants thereof;
g. IV comprises the amino acid sequence of SEQ ID NO: 224 or variants thereof;
h. KP comprises the amino acid sequence of SEQ ID NO: 225 or variants thereof;
i. LZ comprises the amino acid sequence of SEQ ID NO: 226 or variants thereof; and
j. SA comprises the amino acid sequence of SEQ ID NO: 227 or variants thereof;
and wherein the signal peptides enable the extracellular secretion of polypeptide.

9. An expression vector comprising nucleic acid molecule as defined in claim 6, operably linked to a promoter.

10. The expression vector as claimed in claim 9, wherein the promoter for FTase gene is a constitutive, inducible promoter selected from group comprising AOX1, ADH3, DAS, FLD1, LRA3, THI11, GAP, YPT1, TEF1, GCw14 and PGK1.

11. The expression vector as claimed in claim 9, wherein vector is selected from a group comprising pPICZαA, pPICZαB, pPICZαC, pGAPZαA, pGAPZαB, pGAPZαC, pPIC3, pPIC3.5, pPIC3.5K, PAO815, pPIC9, pPIC9K, pHIL-D2 and pHIL-S1 and expression vectors configured for secretory or intracellular expression of FTase.

12. A recombinant host cell comprising an expression vector as claimed in claim 9, wherein the host cell is selected from group comprising Escherichia coli, Bacillus subtilis, Pseudomonas putida, Corynebacterium glutamicum, Saccharomyces cerevisiae, Pichia pastoris and Hansenula polymorpha.

13. The recombinant host cell as claimed in claim 12, wherein the host cell is selected from a group comprising Pichia pastoris Mut+, Mut S, Mut, Pichia pastoris KM71H, Pichia pastoris KM71, Pichia pastoris SMD1168H, Pichia pastoris SMD1168, Pichia pastoris X33, Pichia pastoris GS115 or any other Pichia pastoris host strain.

14. A method of producing a recombinant host cell capable of expressing modified FTase of Aspergillus sp. as claimed in claim 1, said process comprising the steps of:
a. synthesizing a modified nucleic acid molecule as claimed in claim 6;
b. constructing a vector harboring the modified nucleic acid molecule; and
c. transforming a host cell with the vector of step (b) to obtain a recombinant host cell.

15. The method as claimed 14, wherein the modified nucleic acid molecule comprises at least one or more point mutations as defined in claim 2 or deletion mutations as defined in claim 3 or combinations thereof.

16. A process for expressing modified FTase of Aspergillus sp. as claimed in claim 1, comprising:
a. culturing recombinant host cells capable of expressing FTase of Aspergillus sp. in a suitable fermentation medium to obtain a fermentation broth;
b. harvesting supernatant from the fermentation broth, wherein the supernatant contains recombinant FTase; and
c. purifying recombinant FTases.

17. The process as claimed in claim 16, wherein the fermentation medium is Basal Salt Media.

18. The process as claimed in claim 16, wherein the pH of the fermentation broth is maintained in the range from 4.0 to 7.5, and wherein the temperature of the fermentation broth in maintained in the range from 15° C. to 45° C.

19. A process to identify mutations of amino acids across a FTase protein including the active site, wherein the steps comprising:
a. Simulating the apo structure of FTase enzyme using a bioinformatics tool;
b. Clustering the trajectory based on variation of the root mean squared deviation (RMSD) to identify unique conformations;
c. Identifying the unique conformations that the FTase enzyme adopted during the course of the simulation residues for mutation;
d. Analyzing the identified unique conformations of step (c) for pairwise interactions that each amino acid made with another amino acid;
e. Quantifying the pairwise interactions of the amino acids as an indicative interaction strength by providing appropriate weights for the van der Waals and the hydrogen bonds between the pair of residues;
f. Generating 19 mutations of each amino acid that was found to have larger standard deviation in the interaction strengths through the course of the simulation; and
g. Identifying and selecting stabilizing mutations with similar or better stability as per the −ΔΔG values.

20. The process as claimed in claim 19, wherein the protein is selected from Aspergillus sp. preferably Aspergillus niger, Aspergillus japonicus.


SEQUENCE LISTING (241 sequences)

SEQ ID NO: 1 - protein, 654 aa, Aspergillus niger
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 2 - protein, 654 aa, Aspergillus japonicus
MKLTTTTLALATGAAAAEASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERK

SEQ ID NO: 3 - protein, 967 aa, Aspergillus fijiensis
MHFHPEVVFLGGLGLLMGASSSPLPSRPSTPLPEDSAVALSPNTTTGNPLPDLTHLTITPREKDTNVEIVCHNHVFTRDHVKAAVKQAKRIHEAKEEHPHDYAEFPKPLDRRASDLFKKPDELFQFPLVADGVYDGKADVTGCEFVIVDKNLRCKGAVRYYDDHGEDKGHADACYARKRKDKDGTGKGKGKDKEKRKGGGGAGGGGGHASGHASGGHGGGRTGVIVAGGAHGADEGNYDIDSRELAVGETPGKTSAISSAPFTPQPWKVCSEQDITEVAAAAAAQASAQATYKAPHLPGGKTLLEAIQSRSITMKLTTTTLALATGAAAAEASYHLDTTAPPPTNLSTLPNNTLFYVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPANARSSPLQSGFEILASELERTAVYYQFSNESLVVDRSQTSAAAPTNPGLDSFTESGKLRLFDVIENGQEQVETLDLTVVVDNSVVEVYANGRFALSTWARSWYDNSTQIRFFHNGEGEVQFRNVSVSEGLYNAWPERN

SEQ ID NO: 4 - protein, 654 aa, Aspergillus aculeatus
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 5 - DNA, 1965 bp, Aspergillus niger
  source 1..1965: mol_type = genomic DNA
atgaagctcaccactaccaccctggcgctcgccaccggcgcagcagcagcagaagcctcataccacctggacaccacggccccgccgccgaccaacctcagcaccctccccaacaacaccctcttccacgtgtggcggccgcgcgcgcacatcctgcccgccgagggccagatcggcgacccctgcgcgcactacaccgacccatccaccggcctcttccacgtggggttcctgcacgacggggacggcatcgcgggcgccaccacggccaacctggccacctacaccgatacctccgataacgggagcttcctgatccagccgggcgggaagaacgaccccgtcgccgtgttcgacggcgccgtcatccccgtcggcgtcaacaacacccccaccttactctacacctccgtctccttcctgcccatccactggtccatcccctacacccgcggcagcgagacgcagtcgttggccgtcgcgcgcgacggcggccgccgcttcgacaagctcgaccagggccccgtcatcgccgaccaccccttcgccgtcgacgtcaccgccttccgcgatccgtttgtcttccgcagtgccaagttggatgtgctgctgtcgttggatgaggaggtggcgcggaatgagacggccgtgcagcaggccgtcgatggctggaccgagaagaacgccccctggtatgtcgcggtctctggcggggtgcacggcgtcgggcccgcgcagttcctctaccgccagaacggcgggaacgcttccgagttccagtactgggagtacctcggggagtggtggcaggaggcgaccaactccagctggggcgacgagggcacctgggccgggcgctgggggttcaacttcgagacggggaatgtgctcttcctcaccgaggagggccatgacccccagacgggcgaggtgttcgtcaccctcggcacggaggggtctggcctgccaatcgtgccgcaggtctccagtatccacgatatgctgtgggcggcgggtgaggtcggggtgggcagtgagcaggagggtgccaaggtcgagttctccccctccatggccgggtttctggactgggggttcagcgcctacgctgcggcgggcaaggtgctgccggccagctcggcggtgtcgaagaccagcggcgtggaggtggatcggtatgtctcgttcgtctggttgacgggcgaccagtacgagcaggcggacgggttccccacggcccagcaggggtggacggggtcgctgctgctgccgcgcgagctgaaggtgcagacggtggagaacgtcgtcgacaacgagctggtgcgcgaggagggcgtgtcgtgggtggtgggggagtcggacaaccagacggccaggctgcgcacgctggggatcacgatcgcccgggagaccaaggcggccctgctggccaacggctcggtgaccgcggaggaggaccgcacgctgcagacggcggccgtcgtgccgttcgcgcaatcgccgagctccaagttcttcgtgctgacggcccagctggagttccccgcgagcgcgcgctcgtccccgctccagtccgggttcgaaatcctggcgtcggagctggagcgcacggccatctactaccagttcagcaacgagtcgctggtcgtcgaccgcagccagactagtgcggcggcgcccacgaaccccgggctggatagctttactgagtccggcaagttgcggttgttcgacgtgatcgagaacggccaggagcaggtcgagacgttggatctcactgtcgtcgtggataacgcggttgtcgaggtgtatgccaacgggcgctttgcgttgagcacctgggcgagatcgtggtacgacaactccacccagatccgcttcttccacaacggcgagggcgaggtgcagttcaggaatgtctccgtgtcggaggggctctataacgcctggccggagagaaattga

SEQ ID NO: 6 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 7 - DNA, 1965 bp, Aspergillus japonicus
  source 1..1965: mol_type = genomic DNA
atgaagctcaccactaccaccctggcgctcgccaccggtgcagcagcagcagaagcctcataccacctggacaccacggccccgccgccgaccaacctcagcaccctccccaacaacaccctcttccacctgtggcggccgcgcgcgcacatcctgcccgccgagggccagatcggcgacccctgcgcgcactacaccgacccatccaccggcctcttccacgtggggttcctgcacgacggggacggcatcgcgggcgccaccacggccaacctggccacctacaccgacacctccgataacgggagcttcctgatccagccgggcgggaagaacgaccccgtcgccgtgttcgacggcgccgtcatccccgtcggcgtcaacaacacccccaccttactctacacctccgtctccttcctgcccatccactggtccatcccctacacccgcggcagcgagacgcagtcgttggccgtcgcgcgcgacggcggccgccgcttcgacaagctcgaccagggccccgtcatcgccgaccaccccttcgccgtcgacgtcaccgccttccgcgatccgtttgtcttccgcagtgccaggttggatgtgctgctgtcgttggatgaggaggtggcgcggaatgagacggccgtgcagcaggctgtcgatggctggaccgagaagaacgccccctggtatgtcgcggtttctggcggggtgcacggcgtcgggcccgcgcagttcctctaccgccagaacggcgggaacgcttccgagttccagtactgggagtacctcggggagtggtggcaggaggcgaccaactccagctggggcgacgagggcacctgggccgggcgctgggggttcaacttcgagacggggaatgtgctcttcctcaccgaggagggccatgacccccagacgggcgaggtgttcgtcaccctcggcacggaggggtctggcctgcccatcgtgccgcaggtctccagtatccacgatatgctgtgggcggcgggtgaggtcggggtgggcagtgagcaggagggtgccaaggtcgagttctccccctccatggccgggtttctggactgggggttcagcgcctacgctgcggcgggcaaggtgctgccggccagctcggcggtgtcgaagaccagcggcgtggaggtggatcggtatgtctcgttcgtctggttgacgggcgaccagtacgagcaggcggacgggttccccacggctcagcaggggtggacggggtcgctgctgctgccgcgcgagctgaaggtgcagacggtggagaacgtcgtcgacaacgagctggtgcgcgaggagggcgtgtcgtgggtggtgggggagtcggacaaccagacggccacgcttcgcacgctggggatcacgatcgcccgggagaccaaggcggccctgctggccaacggctcggtgaccgcggaggaggaccgcacgctgcagacggcggccgtcgtgccgttcgcgcaatcgccgagctccaagttcttcgtgctgacggcccagctggagttccccgcgagcgcgcgctcgtccccgctccagtccgggttcgaaatcctggcgtcggagctggagcgcacggccatctactaccagttcagcaacgagtcgctggtcgtcgaccgcagccagaccagtgcggcggcgcccacgaaccccgggctggatagctttactgagtccggcaagttgcggttgttcgacgtgatcgagaacggccaggagcaggtcgagacgttggatctcactgtcgtcgtggataacgcggttgtcgaggtgtatgccaacgggcgctttgcgttgagcacctgggcgagatcgtggtacgacaactccacccagatccgcttcttccacaacggcgagggcgaggtgcagttcaggaatgtctccgtgtcggaggggctctataacgcctggccggagagaaagtga

SEQ ID NO: 8 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatttgtggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctagattggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctactttgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaagtga

SEQ ID NO: 9 - DNA, 1965 bp, synthetic construct
  source 1..1965: mol_type = other DNA
atgaaattgactactactactttggctttggctactggtgctgctgctgctgaagcttcttaccatttggatactactgctccacctccaactagattgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 10 - protein, 654 aa, synthetic construct
MKLTTTTLALATGAAAAEASYHLDTTAPPPTRLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 11 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgttttacgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 12 - protein, 654 aa, synthetic construct
MKLTTTTLALATGAAAAEASYHLDTTAPPPTNLSTLPNNTLFYVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 13 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttgttgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 14 - protein, 654 aa, synthetic construct
MKLTTTTLALATGAAAAEASYHLDTTAPPPTNLSTLPNNTLFVVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 15 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttttggtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 16 - protein, 654 aa, synthetic construct
MKLTTTTLALATGAAAAEASYHLDTTAPPPTNLSTLPNNTLFLVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 17 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattccttttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 18 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPFGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 19 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgctaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 20 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGANNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 21 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatacttacactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 22 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTYTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 23 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaagacaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 24 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSERQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 25 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccaccagctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 26 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPPAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 27 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccaagagctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 28 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPRAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 29 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccagctgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 30 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPAAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 31 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccagatgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 32 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPDAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 33 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccagttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 34 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPVAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 35 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccaactgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 36 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPTAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 37 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtgtttggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 38 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGVWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 39 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaatttggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 40 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEFGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 41 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaacatggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 42 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEHGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 43 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaattgggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 44 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFELGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 45 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaagaggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 46 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFERGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 47 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaatctggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 48 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFESGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 49 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaatggggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 50 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEWGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 51 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccattggtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 52 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPLVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 53 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccaaacgtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 54 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPNVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 55 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggttttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 56 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGFGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 57 - DNA, 1872 bp, synthetic construct
  source 1..1872: mol_type = other DNA
aatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggtgttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 58 - protein, 623 aa, synthetic construct
NLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 59 - DNA, 492 bp, synthetic construct
  source 1..492: mol_type = other DNA
aatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttga

SEQ ID NO: 60 - protein, 163 aa, synthetic construct
NLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFV

SEQ ID NO: 61 - DNA, 585 bp, synthetic construct
  source 1..585: mol_type = other DNA
atgaaattgactactactactttggctttggctactggtgctgctgctgctgaagcttcttaccatttggatactactgctccacctccaactaatttgtctactttgcctaacaacactttgtttcatgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttga

SEQ ID NO: 62 - protein, 194 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFV

SEQ ID NO: 63 - DNA, 1965 bp, synthetic construct
  source 1..1965: mol_type = other DNA
atgaaattgactactactactttggctttggctactggtgctgctgctgctgaagcttcttaccatttggatactactgctccacctccaactaatttgtctactttgcctaacaacactttgttttacgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggttttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 64 - protein, 654 aa, synthetic construct
MKLTTTTLALATGAAAAEASYHLDTTAPPPTNLSTLPNNTLFYVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGFGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 65 - DNA, 1872 bp, synthetic construct
  source 1..1872: mol_type = other DNA
aatttgtctactttgcctaacaacactttgttttacgtttggagaccaagagcccatattttgccagctgaaggtcaaattggagatccatgtgctcactacactgatccatctactggtttgtttcatgttggtttcttgcacgatggagatggtattgctggtgctactactgctaatttggctacttatactgatacttctgataacggttctttcttgattcaaccaggtggtaaaaacgatccagttgctgttttcgatggtgctgttattcctgttggtgttaacaatactccaactttgttgtacacttctgtttctttcttgcctattcattggtctattccatatactagaggttctgaaactcaatctttggctgttgctagagatggtggtagaagattcgataaattggatcaaggtcctgttattgctgatcacccatttgctgttgatgttactgctttcagagatccttttgtttttagatccgctaagttggatgttttgttgtctttggatgaagaggttgctagaaatgagactgctgttcaacaagctgttgatggttggactgaaaagaacgctccttggtacgttgctgtttctggtggtgttcatggtgttggtccagctcaatttttgtatagacaaaacggtggtaatgcttctgaattccaatactgggaatatttgggtgaatggtggcaagaagctactaattcttcttggggagatgagggtacttgggctggtagatggggttttaacttcgaaactggtaacgttttgtttttgactgaagagggtcacgatccacaaactggagaggttttcgttactttgggtactgaaggttctggtttgcctattgttccacaagtttcttctattcacgatatgttgtgggctgctggtgaagttggttttggttctgaacaagagggtgctaaggttgaattttctccttctatggctggtttcttggattggggtttctctgcttacgctgctgctggtaaagttttgccagcttcttctgctgtttctaaaacttctggtgttgaggttgatagatacgtttcttttgtttggttgactggagatcaatatgaacaagctgatggtttccctactgctcaacaaggttggactggttctttgttgttgccaagagaattgaaagttcaaactgttgagaacgttgttgataatgaattggttagagaagagggtgtttcttgggttgttggagagtctgataatcaaactgctagattgagaactttgggtattactattgctagagaaactaaggctgctttgttggctaacggttctgttactgctgaagaggatagaactttgcaaactgctgctgttgttcctttcgctcaatctccatcttctaagtttttcgttttgactgctcaattggagtttcctgcttctgctagatcctctccattgcaatctggtttcgaaattttggcttctgaattggagagaactgctatctactaccaattctctaacgagtctttggttgttgatagatcccaaacttctgctgctgctcctactaacccaggtttggattcttttactgagtctggtaaattgagattgttcgatgttatcgaaaacggtcaagaacaagttgagactttggatttgactgttgttgttgataacgctgttgttgaagtttacgctaatggtagatttgctttgtctacttgggctagatcctggtacgataactctactcaaatcagatttttccacaatggtgaaggagaggttcaattcagaaacgtttctgtttctgagggtttgtataacgcttggccagaaagaaattga

SEQ ID NO: 66 - protein, 623 aa, synthetic construct
NLSTLPNNTLFYVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGFGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 67 - protein, 654 aa, synthetic construct
MKLTTTTLALATGAAAAEASYHLDTTAPPPTNLSTLPNNTLFRVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 68 - protein, 654 aa, synthetic construct
MKLTTTTLALATGAAAAEASYHLDTTAPPPTNLSTLPNNTLFTVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 69 - protein, 654 aa, synthetic construct
MKLTTTTLALATGAAAAEASYHLDTTAPPPTNLSTLPNNTLFMVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 70 - protein, 654 aa, synthetic construct
MKLTTTTLALATGAAAAEASYHLDTTAPPPTNLSTLPNNTLFIVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 71 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTWTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 72 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEKQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 73 - protein, 654 aa, synthetic construct
MKLTTTTLALATGAAAAEASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 74 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGYRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 75 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGNRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 76 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPLAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 77 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPWAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 78 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPNAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 79 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPKAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 80 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPQAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 81 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFKSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 82 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 83 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVFGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 84 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEWQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 85 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGLGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 86 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSRVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 87 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSLVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 88 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNERVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 89 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQERVETLDLTVVVDNAVVEVYANGRFALSTWARSWYDNSTQIRFFHNGEGEVQFRNVSVSEGLYNAWPERN

SEQ ID NO: 90 - protein, 654 aa, synthetic construct
MKLTTTTLALATGAAAAEASYHLDTTAPPPTQLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 91 - protein, 654 aa, synthetic construct
MKLTTTTLALATGAAAAEASYHLDTTAPPPTVLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 92 - protein, 654 aa, synthetic construct
MKLTTTTLALATGAAAAEASYHLDTTAPPPTNLSTLPNNTLFWVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 93 - protein, 654 aa, synthetic construct
MKLTTTTLALATGAAAAEASYHLDTTAPPPTNLSTLPNNTLFEVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 94 - protein, 654 aa, synthetic construct
MKLTTTTLALATGAAAAEASYHLDTTAPPPTNLSTLPNNTLFDVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 95 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 96 - protein, 654 aa, synthetic construct
MKLTTTTLALATGAAAAEASYHLDTTAPPPTNLSTLPNNTLFQVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 97 - protein, 654 aa, synthetic construct
MKLTTTTLALATGAAAAEASYHLDTTAPPPTNLSTLPNNTLFFVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 98 - protein, 654 aa, synthetic construct
MKLTTTTLALATGAAAAEASYHLDTTAPPPTNLSTLPNNTLFSVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 99 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTRTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 100 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSELQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 101 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEVQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 102 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGKRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 103 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGHRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 104 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPEAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 105 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSIVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 106 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEFVETLDLTVVVDNAVVEVYANGRFALSTWARSWYDNSTQIRFFHNGEGEVQFRNVSVSEGLYNAWPERN

SEQ ID NO: 107 - protein, 654 aa, synthetic construct
MKLTTTTLALATGAAAAEASYHLDTTAPPPTDLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 108 - protein, 654 aa, synthetic construct
MKLTTTTLALATGAAAAEASYHLDTTAPPPTILSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 109 - protein, 654 aa, synthetic construct
MKLTTTTLALATGAAAAEASYHLDTTAPPPTYLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 110 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERK

SEQ ID NO: 111 - protein, 654 aa, synthetic construct
MKLTTTTLALATGAAAAEASYHLDTTAPPPTKLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 112 - protein, 654 aa, synthetic construct
MKLTTTTLALATGAAAAEASYHLDTTAPPPTNLSTLPNNTLFAVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 113 - protein, 654 aa, synthetic construct
MKLTTTTLALATGAAAAEASYHLDTTAPPPTNLSTLPNNTLFNVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 114 - protein, 654 aa, synthetic construct
MKLTTTTLALATGAAAAEASYHLDTTAPPPTNLSTLPNNTLFKVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 115 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGINNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 116 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGMNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 117 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTHTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 118 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTFTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 119 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTQTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 120 - protein, 654 aa, unidentified
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPLLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 121 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPMLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 122 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPVLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 123 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPILLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 124 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEIQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 125 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERK

SEQ ID NO: 126 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEWQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 127 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEYQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 128 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSEMQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 129 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGLRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 130 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGQRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 131 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADYPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 132 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADRPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 133 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPMAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 134 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPHAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 135 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPSAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 136 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWWVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 137 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVRGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 138 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVKGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 139 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEYQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 140 - protein, 654 aa, synthetic construct
MKLTTTTLALATGAAAAEASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERK

SEQ ID NO: 141 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGIWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 142 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGYPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 143 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPQVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 144 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGQGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 145 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGKGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 146 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGYGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 147 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGIGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 148 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSVVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 149 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSKVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 150 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSPVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 151 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSQVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 152 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSTVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 153 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSYVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 154 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAISKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 155 - protein, 654 aa, synthetic construct
MKLTTTTLALATGAAAAEASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERK

SEQ ID NO: 156 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSALSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 157 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSYTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 158 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEKVETLDLTVVVDNAVVEVYANGRFALSTWARSWYDNSTQIRFFHNGEGEVQFRNVSVSEGLYNAWPERN

SEQ ID NO: 159 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEYVETLDLTVVVDNAVVEVYANGRFALSTWARSWYDNSTQIRFFHNGEGEVQFRNVSVSEGLYNAWPERN

SEQ ID NO: 160 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEVVETLDLTVVVDNAVVEVYANGRFALSTWARSWYDNSTQIRFFHNGEGEVQFRNVSVSEGLYNAWPERN

SEQ ID NO: 161 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEWVETLDLTVVVDNAVVEVYANGRFALSTWARSWYDNSTQIRFFHNGEGEVQFRNVSVSEGLYNAWPERN

SEQ ID NO: 162 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTRFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 163 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRKPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 164 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRRPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 165 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPGVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 166 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPHVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 167 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPMVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 168 - protein, 654 aa, synthetic construct
MKLTTTTLALATGAAAAEASYHLDTTAPPPTNLSTLPNNTLFHLWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 169 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYERADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 170 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEIGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 171 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEKGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 172 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTFFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 173 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTQFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 174 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFREPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 175 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRFPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 176 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRQPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 177 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAEAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 178 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAMAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 179 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYWQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 180 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFLFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 181 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFMFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 182 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFRFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 183 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPAVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 184 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPDVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 185 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPEVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 186 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPFVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 187 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPRVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 188 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYELADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 189 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEMGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 190 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFENGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 191 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEQGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 192 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEVGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 193 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFEYGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 194 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTHFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 195 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTKFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 196 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRAPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 197 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRNPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 198 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRWPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 199 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYALAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 200 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAVAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 201 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYFQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 202 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYGQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 203 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYLQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 204 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYNQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 205 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVYDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 206 - protein, 654 aa, synthetic construct; identical to SEQ ID NO: 1

SEQ ID NO: 207 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFWHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 208 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFKFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 209 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFQFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 210 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPCVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 211 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPIVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 212 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPKVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 213 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPVVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 214 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEKADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 215 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEMADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 216 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVNSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 217 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVTSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 218 - protein, 654 aa, synthetic construct
MKLTTTTLALATGAAAAEASYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSAKLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQWEQADGFPTAQQGWTGSLLLPRELKVQTVENVVDNELVREEGVSWVVGESDNQTARLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 219 - protein, 81 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVS

SEQ ID NO: 220 - protein, 19 aa, Saccharomyces cerevisiae
MRFPSIFTAVLFAASSALA

SEQ ID NO: 221 - protein, 20 aa, Aspergillus niger
MVAWWSLFLYGLQVAAPALA

SEQ ID NO: 222 - protein, 18 aa, Aspergillus awamori
MSFRSLLALSGLVCSGLA

SEQ ID NO: 223 - protein, 16 aa, Kluyveromyces maxianus
MKLAYSLLLPLAGVSA

SEQ ID NO: 224 - protein, 19 aa, Saccharomyces cerevisiae
MLLQAFLFLLAGFAAKISA

SEQ ID NO: 225 - protein, 26 aa, Saccharomyces cerevisiae
MTKPTQVLVRSVSILFFITLLHLVVA

SEQ ID NO: 226 - protein, 26 aa, Gallus gallus
MLGKNDPMCLVLVLLGLTALLGICQG

SEQ ID NO: 227 - protein, 18 aa, Homo sapiens
MKWVTFISLLFLFSSAYS

SEQ ID NO: 228 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 229 - protein, 89 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 230 - protein, 23 aa, synthetic construct
MRFPSIFTAVLFAASSALALEKR

SEQ ID NO: 231 - protein, 24 aa, synthetic construct
MVAWWSLFLYGLQVAAPALALEKR

SEQ ID NO: 232 - protein, 22 aa, synthetic construct
MSFRSLLALSGLVCSGLALEKR

SEQ ID NO: 233 - protein, 20 aa, synthetic construct
MKLAYSLLLPLAGVSALEKR

SEQ ID NO: 234 - protein, 23 aa, synthetic construct
MLLQAFLFLLAGFAAKISALEKR

SEQ ID NO: 235 - protein, 30 aa, synthetic construct
MTKPTQVLVRSVSILFFITLLHLVVALEKR

SEQ ID NO: 236 - protein, 30 aa, synthetic construct
MLGKNDPMCLVLVLLGLTALLGICQGLEKR

SEQ ID NO: 237 - protein, 22 aa, synthetic construct
MKWVTFISLLFLFSSAYSLEKR

SEQ ID NO: 238 - protein, 6 aa, Aspergillus niger
QIGDPC

SEQ ID NO: 239 - protein, 14 aa, Aspergillus niger
DGAVIPVGVNNTPT

SEQ ID NO: 240 - protein, 10 aa, Aspergillus niger
SGLPIVPQVS

SEQ ID NO: 241 - protein, 16 aa, Aspergillus niger
GDQYEQADGFPTAQQG